SEQUENCE LISTING (31 sequences)

SEQ ID NO: 1 - DNA, 3580 bp, Mus musculus
agtttagtagaggccccggtggccggtgctcactgggctcagggccagtccacccgctcaacgcgctctagccagccatgcgtcccgcggccgctaaggtacccaagtggctgcttctcgcactgagcgctctactaccacagtggcccgcagccagtgcctgggagctcacgatcctgcacacaaacgacgtgcacagccggctagagcagaccagcgatgactccaccaagtgcctcaacgccagcctgtgtgtgggcggcgtggcccggctctttaccaaggtgcagcagatccgcaaggaagaacccaacgtgctgtttttggatgccggagaccagtaccagggcaccatctggttcaccgtttacaaaggccttgaagtggcacacttcatgaacatcctgggctacgatgctatggcactgggaaatcatgaatttgataacggtgtggaaggactgattgatcccctcctcagaaacgttaaatttccaattctgagcgcaaacattaaggcacgggggcctctagcacatcagatatctggactttttctgccatctaaagttctctctgttggcggtgaggttgtggggattgttggatatacttcaaaggaaacccctttcctctcaaatccagggacaaatttagtctttgaagatgaaatctctgcattgcagcctgaagtagataaactaaagacgctaaatgtgaataagatcatcgccctggggcactctggttttgagatggacaaacttatcgctcagaaagttcgaggtgtggacatcgtggtgggaggacactccaacacctttctctacacaggaaatccaccttccaaagaagtgcctgcggggaagtacccattcatagtcaccgcagatgatggacggcaggtgcctgtggtccaggcctatgcctttggcaaatacctgggctacctgaaggttgagtttgatgataaaggcaatgttatcacttcctatggaaatcccattcttctcaacagcagcattcctgaagatgcgaccatcaaagcagacattaaccaatggaggataaaattagataattattctacccaggaactcgggagaacgatcgtctacctggatggctccactcagacgtgccgcttcagggaatgcaacatgggaaacctgatctgtgatgccatgattaacaacaacctcagacacccagatgaaatgttttggaaccacgtgtccatgtgcattgtaaacggaggtggcatccggtcccccattgatgagaagaacaatggtaccatcacctgggagaacctggctgctgtgctgccctttggagggacatttgacctcgtccaattaaaagggtccaccctgaagaaggcttttgagcacagcgtgcatcgctatggccagtccaccggagagttcctgcaagtgggtggaatccatgtggtgtacgatattaaccgaaagccctggaacagagtggtccaattagaagttctctgcaccaagtgtcgagtgcccatctatgagcctcttgaaatggataaagtgtataaagtgaccctcccaagctatctggccaacggtggagatggattccagatgataaaagatgaattactaaagcatgactctggtgatcaagatatcagcgtggtttctgaatacatctcaaaaatgaaagtagtttacccagccgttgaagggcggatcaagttctctgcagcaagtcattaccagggaagctttcctttagtaattctttcattttgggcaatgatccttattttgtaccaataacagggagtctccttgtccttgatgtcaaactacatttttcttccagtgatattcatatctgcctctggagacctggctttgtaacagcactcatcatcctcaaggttcctagcagatgttcttcacaaggaagagactgtaacatcatttgttggggccagcaactcagtgagcagatagaaagtcacagtgaaccaacagggtccttctggcagggagtgggtaaggggaaacaactagatgtagcttgcatagccacataacacatctggttaccatttccctttctattcatttctaatccatcaaacaattgatgtttacatacaacttcatcatcgccagctctggtggcacatgcctgtggtcacggcacttggcagggaggagaggatggctgcaagttctaggccagcctgacatatgtagagtttcaggccagtcagctagatatcaagactcacacacacaaacaaacattataatttacaagtagatttttgtagacaagtcttatgataagctaagcagaaagggtcgacttgttcaaggtcagacatctccaaataatacatctaagatctgtacctgagtctttttacctcaagtccaacctctcgcctactgtcaagtctcctctcttcctgcggttggtctcagacaacaaatctgtttctgccttccatagtaccctttcttttgggctcttgttgtctctcaggtttgagagagtagctactggacaggattctttcctacaacaccatgtgcctttggtgagtcatagacaaactgtatacagcagataatagattagtccagggactgcaaaaggcagtcagggacaggcaggaaagggaaaggagaatatgaccaggacttacagaagagagtagatgggttccataaacactagttaaggagcagaacatggctccaaagcacaacactggttcttattaactcctgtgcgactccaaaagccctgcctctctccttccttcttcctcacatggaggtgccatgagagaacttcctaaaataaactgtaattctcaacctgcacctgtcctgtccaagatgccaacacggctctcattcaagtcacacatgtacaatccaatattctggaaggcaagagtgaatttaagaagtggtatacataaatgtttgaagtcactgagactcaaatcccacacaaccactgtaaggcatactcaggtcaagacatgagaagaccagcaggacagtgagccctatgggaggcccaagagatacccagactgatgaaatgatctgtgcccacagaacaccagaccaagacagtcctgtgaccaagtgagcatagtcagcactgtacagtagttaagaatccttaactataaaccagcagctagatgtctggacagggaacatgttggtttcctagtgtttacgaatattaaggactcttgacccaaaccgtttaaaattccaatccttttatgaagtttgttgcagtgaaacaatgccttcagttctttttgaatgtgtagattagttataaacagaaatgtcagtaagactgtaaaaaaaaatgaagaaaggtgcatatgaggtatatttttatgcttggcgagtaaataaggagaaatcttatagcataatttttaaagaacatttgtataatttttctaaatatgtgtatatgtatattttatgcagcagtattgagaaatgaccttggactatcttgtaattgtaaattccaaaaataaagctgaagatatagt

SEQ ID NO: 2 - protein, 576 aa, Mus musculus
MRPAAAKVPKWLLLALSALLPQWPAASAWELTILHTNDVHSRLEQTSDDSTKCLNASLCVGGVARLFTKVQQIRKEEPNVLFLDAGDQYQGTIWFTVYKGLEVAHFMNILGYDAMALGNHEFDNGVEGLIDPLLRNVKFPILSANIKARGPLAHQISGLFLPSKVLSVGGEVVGIVGYTSKETPFLSNPGTNLVFEDEISALQPEVDKLKTLNVNKIIALGHSGFEMDKLIAQKVRGVDIVVGGHSNTFLYTGNPPSKEVPAGKYPFIVTADDGRQVPVVQAYAFGKYLGYLKVEFDDKGNVITSYGNPILLNSSIPEDATIKADINQWRIKLDNYSTQELGRTIVYLDGSTQTCRFRECNMGNLICDAMINNNLRHPDEMFWNHVSMCIVNGGGIRSPIDEKNNGTITWENLAAVLPFGGTFDLVQLKGSTLKKAFEHSVHRYGQSTGEFLQVGGIHVVYDINRKPWNRVVQLEVLCTKCRVPIYEPLEMDKVYKVTLPSYLANGGDGFQMIKDELLKHDSGDQDISVVSEYISKMKVVYPAVEGRIKFSAASHYQGSFPLVILSFWAMILILYQ

SEQ ID NO: 3 - DNA, 4086 bp, Homo sapiens
actcctcctctctgcccctcagctcgctcatctttcttcccgccccctctcttttccttctttggttctttgaagtgatgagctagcgcaaccacaaaccatacattccttttgtagaaaaacccgtgcctcgaatgaggcgagactcagagaggacccaggcgcggggcggacccctccaattccttcctcgcgcccccgaaagagcggcgcaccagcagccgaactgccggcgcccaggctccctggtccggccgggatgcggccggtacccgctccccgccgggaacaacctctccactcttcctgcagggagctggtgccagccgacagccgcgccagggccgctccgggtaccagggtcggatcgggtgacgtcgcgaacttgcgcctggccgccaagccggcctccaggctgaagaaggacccgccccggccttgacccgggccccgcccctccagccggggcaccgagccccggccctagctgctcgcccctactcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagccaaaaattctccttgcctttaatgtgtgaaactgcattttttcaagtgagattcaaatctgccttttaggacctggctttgtgacagcaaaaaccatctttacaggctcctagaagctgaaggttagagcattataaaatgaagagacagacatgattactcagggtcagcaacctagtgagttagaaaaaaaattaacatagggccctataaggagaaagccaactatgttaagtttacgtgtccaaattttaatgaaattttactaacaattttaaaccatatttttcttcttcatatccatttctaatccatcaaacagcttatgtttacataaaattttatcattcacaaggaagttttaagcacactgtctcatttgatatccacaacttatttttggtaggaaagagagatgtttttcccacctgtcagatgaaaaaactgaagctcaaaaagggttgacttgaccatacagctaatgctgacagatccaagacctagacctaggtcttttgaactcaagtccagcattctcaactatatcaagttactgttcagaatacttaatatctcctctcttcataattatcaatagccccaagctcatggatgacaaatctctgctttatttcttgtctctattttttcactttatagctcctgttataatagcaagtttaatggtataaacacaggataccatcctctcttgcaacacccatgtgcctttgatgagtcaggtagcaagctgtagtagataatgagaaaggccagaggctgcaaaagacagtcaaaggacacgagagaaaggaaggggaagaacaggactccaggactgttttatattatagaaaagcaagagctaaagagcatttacacatgttaaacagatacttgttaagcatagtgcctgacacacggcattagctgttattttatgagattccatcagctctgcctctgtcctctttcttctaacatgaaggtatcatgagaagagaaccttctaacataagctgtaattctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaagaacaaatacttaacgtgggagtggaaccacatgagcctgctcagctctgcataagtaattcaagaaatgggaggcttcaccttaaaaacagtgtgcaaatggcagctagaggttttgataggaagtatgtttgtttcttagtgtttacaaatattaagtactcttgatacaaaatatacttttaaacttcataacctttttataaaagttgttgcagcaaaataatagcctcggttctatgcatatatggattagctataaaaaatgtcaataagattgtacaaggaaaattagagaaagtcacatttagggtttattttttacacttggccagtaaaatagggtaaatcctattagaattttttaaagaactttttttaagtttcctaaatctgtgtgtgtattgtgaagtggtataagaaatgactttgaaccactttgcaattgtagattcccaacaataaaattgaagataagctctttggtcaaaaaaaaaaaaaaaaaa

SEQ ID NO: 4 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 5 - DNA, 2989 bp, Artificial Sequence
  5′ homologous arm
ggtgacagtgagccagccttatcttcatagcgttttgccccaccctgcttactttaggaacaagagttttataagttggactactcaggcagtgacatcactagctcagcatgccaagggatcactgacttagtatacatgcaggaagtaggttcttttctagataacactagtttgccccaaacccagtgtcttgaaagggagtcagttgacctccagatagcccttatcaaaatgttcttccttcattgtttccttgagtgggtaggagaacggggaatgttgatgctccacttccctgtgtgatcacacaccactgaacattcaataatatcttcaaattatgtatttgcatcttgtgaattcctacagacttgaggatgtagctcggttggtagagtgattgtctggcatacaggaagcccagggttcaatccccactactgtgtgaactgcacagtgacacatgcttttaaccccaacactagaaagacagacacaagaggaaagagctccaggccatagatgacagtacaatgagctggagggcatcttgaactgcacgtaaccttgtcaaagaagaagaaaaagatttctacagttcaattaaagcagatgcaaatgacagggacagaaggacaacaaaatggcttcatgggtcaaggtacttacaagcctcgtgacctgagtgtcatcgctagaacttacattgtgagaaaaatctgccctgaaaattgtcttctgatctttacacatgtgttatatgtgcacactgcacacatacacacgagcgaaattaataaataaatgcatttttttcaattggggctttccccccctcccttttttgagacagggtttctctgtgtagccctggctatcctgttactcattctgtagaccaggctaccctcaaactcagagatttgcttgcctttgcctcacaagcactgggattaaaggtatgtgccatcaccaccaaactaataaaaaaaaatttaaaggaaatgtctacaagaaaggacacaaatggtccataagtttctatttgtgtttcccagttggtttgacatgctgaccatctttcaaaccaactgggaaacacacatagaaactacagtgagatatcacatgtgccataaggtgactatcatttaagacaaaaaaaaaatagagttggtgaagatgtacagagacttttcgttgcatgtgagaacaagcaacatagcaggtccttacttcacctcccaactaaactatttcatcagccgtgaaccctggtctgattctggtttcataccttatgaataagctgctatgaatgtgagcattcaaatacacacctagtgtgaagtgttggcttttgaaagaaaggaaatgctgacacactgagtcttgaaggcattgcaccaagtaaaacaaattccgtcagaaaagaacaactatgaggtccttagaatgatcaaatctatacaaactggtaatgcaatgttgactgccagaggctggaaaggaaggaaatggagaggtggttttggttagttggtagtttagtttgtttgtgttgttgttgttgctgctgctgttgttgaggcaggcttcactaactcactacatgaccagactgttttcctgccgggactcccaagaactgtaattataggtgattgccaccgtgttgtgtgggagtcactgtttaatgagtttacaatgcaatcttggaaagataaaaatgttctgggaaatgaatgatggcaaatatctgtaaacttaggacagtacgcggtaatgcacatcctgtatatatgatgtatattttattgcaattgaaaagcaacttggttgtgggtgagatagggcctcactatgtaccccatactttcaactctcaactcagtctcctgagtgctaggattacaggtatgccccaatgtagtgtgtgtgtgtgtgtgtgtgtgtgtgtgtgtgtgtgtgtaacaatgcagacctggtgcattctggaattgtgtgggcattagataacttctataatgaccagaacacacgggaacctcaccaatactctttcctttctcagaactactagcgtgttgactatacttcattcaaacacaggggttaatccattagacagggaatttgtgctgtggcgatttatatttaatgatttgttcataacaaatatggatccccactattactgctaaaagctttacacaaacacacacacacacacacacacacacaaacacacacacacacaaacacacacacaccacaaaaggtaagggaggagcgccccagagtagctgtcaggtttacccgctaggatccgccttagctttcagagctggccctatgccaccgctgagggtcccaaagattgctttgctcctgagattccagctcgaatacgtggagcggttgcgtcctcaaacctaagtagcgtctcggccggtgtgtcccggtcccaccgaagcaaccttccagcgcacctctcaggcgggctctggacccctctcactcccacgtttgtcttcctttccttcccccccccaccccccccgccccagccccttctttttcggcgaccgagccaccgagcaacacaagccgctgtacactcttctggagaaatcggtgtaatagagagtgaggatccggaacgctgcccctgcagttgtcaccgccgcccgcccggggactgcggcacccagcctctcggacctgctcgctcaccaggcgaacaacctcctctctgttccctgcgggctgcgtgtgctgggccagaggctcccgggtgcctcgggtgccagggtcgcatcgggtgactcggggagtgtgtctgcaagcgggagggcctggggctggaagaggcgggtctgtccccgccccgccctcctctactccgcagtttagtagaggccccggtggccggtgctcactgggctcagggccagtccacccgctcaacgcgctctagccagcc

SEQ ID NO: 6 - DNA, 4849 bp, Artificial Sequence
  3′ homologous arm
aaggtacccaagtggctgcttctcgcactgagcgctctactaccacagtggcccgcagccagtgcctgggagctcacgatcctgcacacaaacgacgtgcacagccggctagagcagaccagcgatgactccaccaagtgcctcaacgccagcctgtgtgtgggcggcgtggcccggctctttaccaaggtgcagcagatccgcaaggaagaacccaacgtgctgtttttggatgccggagaccagtaccagggcaccatctggttcaccgtttacaaaggccttgaagtggcacacttcatgaacatcctgggctacgatgctatggtaaggagaaagttcccggggtaggagtcccagacccagagagagaaaggctggagaactcgctagtggtctgatagtgctcagagatgtacaggtggaactcgacccaggccacctaaggattcattcactaagtgagggcgtctcttagattattttcttaaactgatgttattggggtcccgtagcgaactgagacaattagaagagggaccctactgcctacaggtgcttcagctcgagcactgaaaattatcaagggctggagacccatcccagtctcctccacctctcccatctgctctgtcttccaaacaattcttcatccttactttactgaaaggaaatcgggtcaaaaattattcaggtcaccagcccccaaggaggaaaacacatccgattggctctgaagtccgagcacaatgagctcacctccttaggtcgatttacaggtcagttacttttctgtgaccttatactagttatttattagacctcggtggccaaatctgctatgaaatgcgaagaagaatgttgacctcaaagcactgttgtgcacataatataataatataatctctgtaactgttagttcttgttgctcccaggcactgcctcaaaagagcaagttaaaaaaaaaaaagtcagagagaacccaaacgcggaaaagctgggatttcaggggctcaataaaaaccgattggctgtagagattccgaaagagcctaactggagagataagggggctggggatgcagctccattgttagagtactttcttagaattccaaaggccctggatttgatccctggcatcgcccaaactaggcttgcagggcaatcctggtaaaggttgaaagatccaaagttcagagtcatccttggttacaaaagagaggccagcctagcatgagagagagagagagagagagagagagagagagagagagagagagagagagagagagagattagtcctcacttcccaacactctgttggaggtgatgccttgcttgttgagaagggataaagaataatcaaataaaacataactgagatgtaactgagacgtaaaaattaattgagccaccactcacattcaacccagtgtggatgcctccggtaaaagattctgccctgttctggggaaaaaatcagagacaacaacaacaatcataagggcttagcaagagaaatagcagggcgagttctcagaactctgtgaaaccagagggtgtgggagggaaagtaaggtgcagacagcagactacccactgtgtacaggaacagctcagaggtcagagaagggtgagagctcccttcctactgaatattggcttagagactctcaatgcaagaacggctctaacagtccttaagcctacagagttgtctggggagggccatcatgctcagcagcagcttggcaaaagaaattccgacctcaagaactctgcagtctctcagctttctttttccacagctagacctactcgaagctttgcaagtggtaacggcaatacaaacatttacatattcggattgttaactagctcacgtctctacttcctctcagaagggtggataaacaacgtctgggtttgctttgatttcacatataagccacaactttacagcattttctttttcgaacactctttttcttctcatgtgggtaaatattccttagtaaaaatgcgtccctctttaagacaggatggggccacaacaatgtactacaagttggaacccatcacccttgaatgtttagaatactttttgtgggatggaattgctttcttacaaatgtctatccaagtcattgaagtggggcctgaggaaaaataagctacactggccggtgtcaagagtgggaggagaggggataacagaatctatagctccacttttcagagaggtctttagaacatatgggtggggtgagtcgtgggtggggttcaatacacatgatttggccttcgtggaccatagctttcacggctgtagcatttggttccgctctgcagcctttgtatctcacagaggtttacactgttgtgttgcttgtattttactaagccttgggagcctagtgatcttctactataaaaaattccttgtctcagttccctgctgtgtcttgattctgcgatgctggaaacccagatcagagtgaacagctgctgtagaggggtatctccagttccccatccatggaagccagtgtagcctcggaaattagagcaggagcctccaagacattttgtactctctagacctgccacactcatgagagagttgttctggtggctatggaggaccaggatgaacttgaagacccccttaggttctgctgctaggcagaaactgagagatgacagaagcagccagggagtgtagctgtctatcctagtggaaagactaatgcgccctgagggatgaggagagagacacaccaagctattacacaaaccagctcagaggtcaggaaagcactgtactggtttgggtttttgtttgtttgttctctctctctctctctctctctctctctctctctctcacatccaggagaaaggcagacaggaaaagaactcgaggaagctcagctgtttgcagattctcttattcttttcggcctggttatttctgagcctcatgttccttaactgggaaaggtaacccccagcctatggacatggtacaatgagaaatcccttgctcaattaaagcttagttctctatatctcgggtttgggaggaagaggacgtgacagctgtcatttatgtgggtctgacagccttccctccaccatcttttctaagcaccactgtctaaactgttccttttagaatgtttttcctttttaagttctcagatagcctaaactggtctctaacttgctgtgtggtagaggatgtcgctgaacctctgatcatcctgcctccacctctcaaatgctacttttgtgttcctgtcttggttacttttccattgctgtgacaaaatgctatgactaaggcagcttactaaagaaagcatttaattaggcctatgattgcagagtgttagagttcatgatggcagagcaaaggcgtggtatcaggaacagctgagagctcacatcttgatccacaagtaggaggcagacagagaggggttcactgggaattgtgtgtgtcttctgaagccccaaagccctcctccagtggtacatctcctctaacaaggccacaccttctagatcttcccaaacaattctgccaactgtgttcagactatgtgttcaaacataagagcctatgggctgttctcattcagtccactactgtgccttgcttttatgatttttcccacctcattctacatgtgcagtgcccctgtgtgtgtgtttaggtacacggtgaactcttcacacctcattctctggtcaatttggagatggcttctaatcaaccccccctccccccacgcgcctcatgaattcagctgttctaaatatcttctgctgattccattgtgagaattttcatattcaagtgcaaagttactggtcacaatatgtatgcttggttcctcctttcacaaaggattgccagctgccttcctgaaggcttaataacccaggcaagaacattaatctttgtatcattactccttctttaaaaaaaaaaagatatttttattctcatgtatgtgtaccagatgtgtgctggtgcctgtgaaagccagatcctctctggaaatggagttattggaagctgttagccatatgatataaaggctggacaccaaattctgtcccctacaagagtatcaagtgctcgtaactgttgagccatctccccagccctcatcattgcctatccagcttgtcagtagcttgtgtttttctaataatcggacatatcgaaagttgtttttgataagtaagtttgtgaataatttgggtttcctcttcatcatctgatgtcatgtttacagatttttcttttttcattttttccattggttttgctttatttacaaacactctaaccattgatgtacaatcaattttagacataaatttgatggtgctactatgcctgtcaactttcaactctctgcctcttagaacaaaaacccttcattgggtatattcaaataatgaaaaaaattcctgtttgtctgtctgttttggcctgtgttctggaaaacttatggttcaatttgctactttaaagagcaaattgccaaggctctcagtggaaaagcaatcgctgctgtctctctgccttttaggacatcagcctgtcagaactgcctgttagtttcctctccagcgtgcaggttaatgggggcagaagcagcatatccctggggctcttctctaaagctgggcggtcctgagaaacggtttaatgctgtaagatgcttctgttagggaggagcacaggtatggagacctggacagaagtgaaactaagataagatgaaaatgagatagacccggcagacctaagagaagtttaagctcc

SEQ ID NO: 7 - DNA, 1725 bp, Artificial Sequence
  Fragment of human NT5E DNA
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatag

SEQ ID NO: 8 - DNA, 589 bp, Artificial Sequence
  WPRE sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 9 - DNA, 208 bp, Artificial Sequence
  PolyA signal sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 10 - DNA, 88 bp, Artificial Sequence
  downstream junction of the Neo cassette and the mouse NT5E locus
gaacttcatcagtcaggtacataatggtggatccgatatcaaggtacccaagtggctgcttctcgcactgagcgctctactaccacag

SEQ ID NO: 11 - DNA, 20 bp, Artificial Sequence
  primer
gctcgactagagcttgcgga

SEQ ID NO: 12 - DNA, 25 bp, Artificial Sequence
  primer
tagagccccagttcaaaagcaacct

SEQ ID NO: 13 - DNA, 25 bp, Artificial Sequence
  primer
acaaagcaggtggacagcatcctac

SEQ ID NO: 14 - DNA, 25 bp, Artificial Sequence
  primer
atctgctgaaccttggtgaagagcc

SEQ ID NO: 15 - DNA, 22 bp, Artificial Sequence
  primer
ccttctttttcggcgaccgagc

SEQ ID NO: 16 - DNA, 21 bp, Artificial Sequence
  primer
gctggctagagcgcgttgagc

SEQ ID NO: 17 - DNA, 23 bp, Artificial Sequence
  primer
ggaggaaatggaagcaggccagg

SEQ ID NO: 18 - DNA, 22 bp, Artificial Sequence
  primer
ctagccagtgtcacccccaagg

SEQ ID NO: 19 - DNA, 23 bp, Artificial Sequence
  primer
ggatcggccattgaacaagatgg

SEQ ID NO: 20 - DNA, 23 bp, Artificial Sequence
  primer
cagaagaactcgtcaagaaggcg

SEQ ID NO: 21 - DNA, 21 bp, Artificial Sequence
  primer
ctgcccctgcagttgtcaccg

SEQ ID NO: 22 - DNA, 21 bp, Artificial Sequence
  primer
agctcccaggcactggctgcg

SEQ ID NO: 23 - DNA, 25 bp, Artificial Sequence
  primer
tcctgttggtgatgaagttgtggga

SEQ ID NO: 24 - DNA, 25 bp, Artificial Sequence
  primer
agcataggcctggactacaggaacc

SEQ ID NO: 25 - DNA, 25 bp, Artificial Sequence
  primer
cctcagacgagtcggatctcccttt

SEQ ID NO: 26 - DNA, 24 bp, Artificial Sequence
  primer
ctgcgggccactgtggtagtagag

SEQ ID NO: 27 - DNA, 25 bp, Artificial Sequence
  primer
gacaagcgttagtaggcacatatac

SEQ ID NO: 28 - DNA, 24 bp, Artificial Sequence
  primer
gctccaatttcccacaacattagt

SEQ ID NO: 29 - DNA, 2550 bp, Artificial Sequence
  A fragment sequence
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagatttaaataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 30 - DNA, 80 bp, Artificial Sequence
  WPRE  alpha component
gccacggcggaactcatcgccgcctgccttgcccgctgctggacaggggctcggctgttgggcactgacaattccgtggt

SEQ ID NO: 31 - protein, 524 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

(19) United States
(12) Patent Application Publication
(10) Pub. No.: US 2022/0211019 A1
(43) Pub. Date: Jul. 7, 2022

(54) GENETICALLY MODIFIED NON-HUMAN ANIMAL WITH HUMAN OR CHIMERIC CD73

(71) Applicants: Biocytogen Pharmaceuticals (Beijing) Co., Ltd., Beijing (CN); Biocytogen Jiangsu Co., Ltd., Jiangsu (CN)

(72) Inventors: Yuelei Shen, Beijing (CN); Yanan Guo, Beijing (CN); Yang Bai, Beijing (CN); Rui Huang, Beijing (CN); Chengzhang Shang, Beijing (CN); Meiling Zhang, Beijing (CN); Jiawei Yao, Beijing (CN); Chaoshe Guo, Beijing (CN)

(21) Appl. No.: 17/295,303

(22) PCT Filed: Nov. 20, 2019
(86) PCT No.: PCT/CN2019/119793

§ 371 Date: May 19, 2021

(30) Foreign Application Priority Data
Nov. 20, 2018 (CN) 201811381127.7

(51) Int. Cl. A01K 67/027; C07K 14/705

(52) U.S. Cl. CPC A01K 67/0278; C07K 14/70596; A01K 2207/15; A01K 2267/0331; A01K 2217/15; A01K 2227/105; C07K 2319/00; A01K 2217/072

(57) ABSTRACT

The present disclosure relates to genetically modified non-human animals that express a human or chimeric (e.g., humanized) CD73, and methods of use thereof.

### CLAIM OF PRIORITY

This application claims the benefit of Chinese Patent Application App. No. 201811381127.7, filed on Nov. 20, 2018. The entire contents of the foregoing are incorporated herein by reference.

### TECHNICAL FIELD

This disclosure relates to genetically modified animal expressing human or chimeric (e.g., humanized) CD73, and methods of use thereof.

### BACKGROUND

The traditional drug research and development typically involve in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an animal model with human CD73 or chimeric CD73. The animal model can express human CD73 or chimeric CD73 (e.g., humanized CD73) protein in its body. It can be used in the studies on the function of CD73 gene, and can be used in the screening and evaluation of anti-human CD73 antibodies. In addition, the animal models prepared by the methods described herein can be used in drug screening, pharmacodynamics studies, treatments for immune-related diseases (e.g., autoimmune disease), and cancer therapy for human CD73 target sites; they can also be used to facilitate the development and design of new drugs, and save time and cost. In summary, this disclosure provides a powerful tool for studying the function of CD73 protein and a platform for screening cancer drugs.

In one aspect, the disclosure relates to a genetically-modified, non-human animal whose genome comprises at least one chromosome comprising a sequence encoding a human or chimeric CD73.

In some embodiments, the sequence encoding a human or chimeric CD73 comprises a sequence encoding an amino acid sequence that is at least 70%, 75%, 80%, 85%, 90%, 95%, 99%, or 100% identical to NP 002517.1 (SEQ ID NO: 4) or NP 001191742.1 (SEQ ID NO: 31).

In some embodiments, the sequence encoding a human or chimeric CD73 is operably linked to a Woodchuck Hepatitis Virus (WHP) Posttranscriptional Regulatory Element and/or a polyA (polyadenylation) signal sequence.

In some embodiments, the sequence encoding the human or chimeric CD73 is operably linked to an endogenous promoter around endogenous CD73 transcription start site. In some embodiments, the sequence is contiguously linked to the 5′-UTR of the endogenous CD73.

In some embodiments, the animal is a mammal, e.g., a monkey, a rodent or a mouse. In some embodiments, the animal is a mouse.

In some embodiments, the animal does not express endogenous CD73. In some embodiments, the animal has one or more cells expressing human or chimeric CD73. In some embodiments, the animal expresses a decreased level of human or chimeric CD73 (e.g., as compared to the expression level of CD73 in a wildtype animal). In some embodiments, the animal expresses a decreased level of endogenous CD73 (e.g., as compared to the expression level of CD73 in a wildtype animal).

In one aspect, the disclosure relates to a genetically-modified, non-human animal, wherein the genome of the animal comprises an insertion of a sequence encoding a human CD73 or a chimeric CD73 at an endogenous CD73 gene locus.

In some embodiments, the sequence encoding the human CD73 or the chimeric CD73 is operably linked to the 5′-UTR at the endogenous CD73 locus, and one or more cells of the animal expresses the human CD73 or the chimeric CD73.

In some embodiments, the animal does not express endogenous CD73.

In some embodiments, the animal is a mouse, and the sequence encoding the chimeric CD73 comprises one or more exons selected from the group consisting of exon 1, exon 2, exon 3, exon 4, exon 5, exon 6, exon 7, exon 8 and exon 9 of human CD73 gene.

In some embodiments, the animal is heterozygous with respect to modification at the endogenous CD73 gene locus. In some embodiments, the animal is homozygous with respect to the modification at the endogenous CD73 gene locus.

In one aspect, the disclosure relates to a method for making a genetically-modified, non-human animal, comprising: replacing in at least one cell of the animal, at an endogenous CD73 gene locus, a sequence encoding a region of an endogenous CD73 with a sequence encoding a corresponding region of human CD73.

In some embodiments, the sequence encoding the corresponding region of human CD73 comprises exon 1, exon 2, exon 3, exon 4, exon 5, exon 6, exon 7, exon 8, and/or exon 9, or a part thereof, of a human CD73 gene.

In some embodiments, the sequence encoding the corresponding region of human CD73 encodes a sequence that is at least 50%, 60%, 70%, 80%, or 90% identical to SEQ ID NO: 4 or SEQ ID NO: 31.

In some embodiments, the animal is a mouse, and the endogenous CD73 locus is exon 1, exon 2, exon 3, exon 4, exon 5, exon 6, exon 7, exon 8, and/or exon 9 of the mouse CD73 gene.

In one aspect, the disclosure relates to a non-human animal comprising at least one cell comprising a nucleotide sequence encoding a chimeric CD73 polypeptide, wherein the chimeric CD73 polypeptide comprises at least 50 contiguous amino acid residues that are identical to the corresponding contiguous amino acid sequence of a human CD73. In some embodiments, the animal expresses the chimeric CD73.

In some embodiments, the chimeric CD73 polypeptide comprises a sequence that is at least 80%, 90%, 95%, or 99% identical to SEQ ID NO: 4 or SEQ ID NO: 31.

In some embodiments, the nucleotide sequence is operably linked to the 5′-UTR at the endogenous CD73 locus immediately before the translation start codon.

In some embodiments, the nucleotide sequence is integrated to an endogenous CD73 gene locus of the animal.

In one aspect, the disclosure relates to a method of making a genetically-modified mouse cell that expresses a chimeric CD73, the method comprising: replacing at an endogenous mouse CD73 gene locus, a nucleotide sequence encoding a region of mouse CD73 with a nucleotide sequence encoding a corresponding region of human CD73, thereby generating a genetically-modified mouse cell that includes a nucleotide sequence that encodes the chimeric CD73. In some embodiments, the mouse cell expresses the chimeric CD73.

In some embodiments, the nucleotide sequence encoding the chimeric CD73 is operably linked to an endogenous promoter around endogenous CD73 transcription start site and/or a Woodchuck Hepatitis Virus (WHP) Posttranscriptional Regulatory Element.

In some embodiments, the animal further comprises a sequence encoding an additional human or chimeric protein.

In some embodiments, the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α(SIRPα) or TNF Receptor Superfamily Member 4 (OX40).

In one aspect, the disclosure relates to a method of determining effectiveness of an anti-CD73 antibody for the treatment of cancer, comprising: administering the anti-CD73 antibody to the animal as described herein, wherein the animal has a tumor; and determining the inhibitory effects of the anti-CD73 antibody to the tumor.

In some embodiments, the anti-CD73 antibody inhibits CD73 in catalyzing conversion of AMP to adenosine. In some embodiments, the tumor comprises one or more cancer cells that are injected into the animal.

In some embodiments, determining the inhibitory effects of the anti-CD73 antibody to the tumor involves measuring the tumor volume in the animal.

In some embodiments, the tumor cells are solid tumor cells.

In one aspect, the disclosure relates to a method of determining effectiveness of an anti-CD73 antibody and an additional therapeutic agent for the treatment of a tumor, comprising administering the anti-CD73 antibody and the additional therapeutic agent to the animal as described herein, wherein the animal has a tumor; and determining the inhibitory effects on the tumor.

In some embodiments, the animal further comprises a sequence encoding a human or chimeric programmed cell death protein 1 (PD-1). In some embodiments, the animal further comprises a sequence encoding a human or chimeric cytotoxic T-lymphocyte antigen 4 (CTLA4). In some embodiments, the additional therapeutic agent is an anti-PD-1 antibody, an anti-CTLA4 antibody, or anthracycline.

In some embodiments, the tumor comprises one or more tumor cells that express CD80, CD86, PD-L1 or PD-L2.

In some embodiments, the tumor is caused by injection of one or more cancer cells into the animal.

In some embodiments, determining the inhibitory effects of the treatment involves measuring the tumor volume in the animal.

In some embodiments, the animal has solid tumors, glioma, head and neck cancer, melanoma, thyroid cancer, breast cancer, pancreatic cancer, colon cancer, bladder cancer, ovarian cancer, prostate cancer, or leukemia.

In one aspect, the disclosure relates to a protein comprising an amino acid sequence, wherein the amino acid sequence is one of the following:
(a) an amino acid sequence set forth in SEQ ID NO: 4 or SEQ ID NO: 31;
(b) an amino acid sequence that is at least 90% identical to SEQ ID NO: 4 or SEQ ID NO: 31;
(c) an amino acid sequence that is at least 91%, 92%, 93%, 94%, 95%, 96%, 97%, 98%, or 99% identical to SEQ ID NO: 4 or SEQ ID NO: 31;
(d) an amino acid sequence that is different from the amino acid sequence set forth in SEQ ID NO: 4 or SEQ ID NO: 31 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 4 or SEQ ID NO: 31.

In one aspect, the disclosure relates to a nucleic acid comprising a nucleotide sequence, wherein the nucleotide sequence is one of the following:
(a) a sequence that encodes the protein as described herein;
(b) SEQ ID NO: 3, SEQ ID NO: 7, SEQ ID NO: 10, or SEQ ID NO: 29;
(c) a sequence that is at least 90% identical to SEQ ID NO: 3, SEQ ID NO: 7, or SEQ ID NO: 29;
(d) a sequence that is at least 91%, 92%, 93%, 94%, 95%, 96%, 97%, 98%, or 99% identical to SEQ ID NO: 3, SEQ ID NO: 7, or SEQ ID NO: 29.

In one aspect, the disclosure relates a cell, a tissue, or an animal that has the protein as described herein and/or the nucleic acid as described herein.

In another aspect, the disclosure also provides a genetically-modified, non-human animal whose genome comprise a disruption in the animal's endogenous CD73 gene, wherein the disruption of the endogenous CD73 gene comprises deletion of exon 1, exon 2, exon 3, exon 4, exon 5, exon 6, exon 7, exon 8, and/or exon 9, or part thereof of the endogenous CD73 gene.

In some embodiments, the disruption of the endogenous CD73 gene comprises deletion of one or more exons or part of exons selected from the group consisting of exon 1, exon 2, exon 3, exon 4, exon 5, exon 6, exon 7, exon 8, and exon 9 of the endogenous CD73 gene.

In some embodiments, a stop codon or a polyA (polyadenylation) signal is inserted in one or more exons or part of exons selected from the group consisting of exon 1, exon 2, exon 3, exon 4, exon 5, exon 6, exon 7, exon 8, and exon 9 of the endogenous CD73 gene (e.g., exon 1).

In some embodiments, the disruption of the endogenous CD73 gene further comprises deletion of one or more introns or part of introns selected from the group consisting of intron 1, intron 2, intron 3, intron 4, intron 5, intron 6, intron 7, and intron 8 of the endogenous CD73 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 800, 1000, 1500, 2000, 2500, 3000, 3500, 4000 or more nucleotides.

In some embodiments, the disruption of the endogenous CD73 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, exon 8, and/or exon 9 (e.g., deletion of at least 300 nucleotides of exon 1).

In some embodiments, the mice described in the present disclosure can be mated with the mice containing other human or chimeric genes (e.g., chimeric PD-1, chimeric PD-L1, chimeric CTLA-4, or other immunomodulatory factors), so as to obtain a mouse expressing two or more human or chimeric proteins. The mice can also, e.g., be used for screening antibodies in the case of a combined use of drugs, as well as evaluating the efficacy of the combination therapy.

In another aspect, the disclosure further provides methods of determining toxicity of an agent (e.g., a CD73 antagonist or agonist). The methods involve administering the agent to the animal as described herein; and determining weight change of the animal. In some embodiments, the method further involve performing a blood test (e.g., determining red blood cell count).

In one aspect, the disclosure relates to a targeting vector, including a) a DNA fragment homologous to the 5′ end of a region to be altered (5′ arm), which is selected from the CD73 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CD73 gene genomic DNAs in the length of 100 to 10,000 nucleotides.

In some embodiments, a) the DNA fragment homologous to the 5′ end of a region to be altered (5′ arm/receptor) is selected from the nucleotide sequences that have at least 90% homology to the NCBI accession number NC 000075.6; c) the DNA fragment homologous to the 3′ end of the region to be altered (3′ arm/receptor) is selected from the nucleotide sequences that have at least 90% homology to the NCBI accession number NC 000075.6.

In some embodiments, a) the DNA fragment homologous to the 5′ end of a region to be altered (5′ arm/receptor) is selected from the nucleotides from the position 88324697 to the position 88327685 of the NCBI accession number NC 000075.6; c) the DNA fragment homologous to the 3′ end of the region to be altered (3′ arm/receptor) is selected from the nucleotides from the position 82327704 to the position 88332552 of the NCBI accession number NC 000075.6.

In some embodiments, a length of the selected genomic nucleotide sequence is more than 1 kb, 2 kb, 3 kb, 3.5 kb, 4 kb, 4.5 kb, 5 kb, 5.5 kb, or 6 kb. In some embodiments, the region to be altered is exon 1, exon 2, exon 3, exon 4, exon 5, exon 6, exon 7, exon 8, and/or exon 9 of mouse CD73 gene (e.g., exon 1).

In some embodiments, the sequence of the 5′ arm is shown in SEQ ID NO: 5. In some embodiments, the sequence of the 3′ arm is shown in SEQ ID NO: 6.

In some embodiments, the targeting vector further includes a selectable gene marker.

In some embodiments, the target region is derived from human. In some embodiments, the target region is a part or entirety of the nucleotide sequence of a humanized CD73. In some embodiments, the nucleotide sequence is shown as one or more of exon 1, exon 2, exon 3, exon 4, exon 5, exon 6, exon 7, exon 8, exon 9 of the human CD73.

In some embodiments, the nucleotide sequence of the human CD73 encodes the human CD73 protein with the NCBI accession number NP_002517.1 (SEQ ID NO: 4) or NP_001191742.1 (SEQ ID NO: 31).

In some emboldens, the nucleotide sequence of the human CD73 is selected from the nucleotides from the position 557 to position 2281 of NM_002526.3 (SEQ ID NO: 3). The disclosure also relates to a cell including the targeting vector as described herein.

The disclosure also relates to a method for establishing a genetically-modified non-human animal expressing two human or chimeric (e.g., humanized) genes. The method includes the steps of

(a) using the method for establishing a CD73 gene humanized animal model to obtain a CD73 gene genetically modified humanized mouse;

(b) mating the CD73 gene genetically modified humanized mouse obtained in step (a) with another humanized mouse, and then screening to obtain a double humanized mouse model.

In some embodiments, in step (b), the CD73 gene genetically modified humanized mouse obtained in step (a) is mated with a PD-1 or CTLA4 humanized mouse to obtain a CD73 and PD-1 double humanized mouse model or a CD73 and CTLA4 double humanized mouse model.

The disclosure also relates to non-human mammal generated through the methods as described herein.

In some embodiments, the genome thereof contains human gene(s). In some embodiments, the non-human mammal is a rodent. In some embodiments, the non-human mammal is a mouse.

In some embodiments, the non-human mammal expresses a protein encoded by a humanized CD73 gene.

The disclosure also relates to an offspring of the non-human mammal.

In another aspect, the disclosure relates to a tumor bearing non-human mammal model, characterized in that the non-human mammal model is obtained through the methods as described herein. In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to a CD73 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the CD73 gene function, human CD73 antibodies, the drugs or efficacies for human CD73 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing the mouse NT5E gene locus and the human NT5E gene locus.

FIG. 2 is a schematic diagram showing humanized NT5E gene locus in mice.

FIG. 3 is a schematic diagram showing an NT5E gene targeting strategy.

FIG. 4 is an image showing Southern blot results. WT indicates wild-type.

FIG. 5 is a schematic diagram showing the FRT recombination process that removes NeoR.

FIG. 6A shows PCR identification results for F1 generation mice, wherein primer pairs WT-F1 and WT-R2 were used to amplify wild-type mouse NT5E gene exon 1 fragment. WT is wild-type; H₂O is a blank control; M is the Marker; PC1 and PC2 are positive controls.

FIG. 6B shows PCR identification results for F1 generation mice, wherein primer pairs Mut-F2 and Mut-R2 were used to amplify the human sequence fragment of the modified NT5E gene exon 1 to verify the correct insertion of the recombinant vector into the genomic locus. WT is wild-type; H₂O is a blank control; M is the Marker; PC1 and PC2 are positive controls.

FIG. 6C shows PCR identification results for F1 generation mice, wherein primer pairs Frt-F and Frt-R were used to amplify the Neo fragment to verify whether NeoR was removed. WT is wild-type; H₂O is a blank control; M is the Marker; PC1 and PC2 are positive controls.

FIG. 6D shows PCR identification results for F1 generation mice, wherein primer pairs Frt-F2 and Frt-R2 were used to confirm the presence of the Flp fragment. WT is wild-type; H₂O is a blank control; M is the Marker; PC1 and PC2 are positive controls.

FIG. 7A is a graph showing the flow cytometry analysis result of wild-type C57BL/6 mice, wherein cells were stained by APC conjugated anti-mouse CD73 antibody (mCD73 APC) and PerCP/Cy55 conjugated anti-mouse TCR beta chain antibody (mTcRβ PerCP).

FIG. 7B is a graph showing the flow cytometry analysis result of NT5E gene humanized heterozygous mice, wherein cells were stained by APC conjugated anti-mouse CD73 antibody (mCD73 APC) and PerCP/Cy55 conjugated anti-mouse TCR beta chain antibody (mTcRβ PerCP).

FIG. 7C is a graph showing the flow cytometry analysis result of wild-type C57BL/6 mice, wherein cells were stained by PE conjugated anti-human CD73 antibody (hCD73 PE) and PerCP/Cy55 conjugated anti-mouse TCR beta chain antibody (mTcRβ PerCP).

FIG. 7D is a graph showing the flow cytometry analysis result of NT5E gene humanized heterozygous mice, wherein cells were stained by PE conjugated anti-human CD73 antibody (hCD73 PE) and PerCP/Cy55 conjugated anti-mouse TCR beta chain antibody (mTcRβ PerCP).

FIG. 8 shows the alignment between mouse CD73 amino acid sequence (NP_035981.1; SEQ ID NO: 2) and human CD73 amino acid sequence (NP_002517.1; SEQ ID NO: 4).

FIG. 9. The average weight of humanized NT5E gene heterozygous mice that were injected with mouse colon cancer cells MC38 and were treated with an anti-CD73 antibody MEDI9447. There was no significant difference in average body weight between the G1 control group and G2 MEDI9447 treatment group.

FIG. 10. The percentage change of average weight of humanized NT5E gene heterozygous mice that were injected with mouse colon cancer cells MC38 and were treated with an anti-CD73 antibody MEDI9447.

FIG. 11. The average tumor volume in humanized NT5E gene heterozygous mice that were injected with mouse colon cancer cells MC38 and were treated with an anti-CD73 antibody MEDI9447. The tumor volume in G2 MEDI9447 treatment was significantly smaller than the G1 control group.

### DETAILED DESCRIPTION

This disclosure relates to transgenic non-human animal with human or chimeric (e.g., humanized) CD73, and methods of use thereof.

Immune checkpoint inhibitors have revolutionized clinical oncology and strongly renewed the interest for immune-based treatments of cancer. While immune checkpoint blockade therapy is very effective in a fraction of cancer patients, sometimes leading to complete and long-lasting remission, the majority of patients fail to respond to this therapy. Multiple redundant and non-redundant immunosuppressive pathways active in the tumor microenvironment (TME) can at least partly explain failure to current immune checkpoint therapy.

The conversion of extracellular adenosine triphosphate (ATP) into extracellular adenosine is a form of immune checkpoint that interferes with anti-tumor immune responses by preventing the pro-inflammatory action of ATP and by engaging adenosine signaling in immune cells and endothelial cells. Targeted blocked of the main effectors of this pathway, including the ecto-5′-nucleotidase CD73 responsible for hydrolyzing of AMP to adenosine, the ATPase CD39 responsible for hydrolyzing ATP and adenosine diphosphate (ADP) to adenosine monophosphate (AMP), and the adenosine receptors ADORA2A (A2a) and ADORA2B (A2b) responsible for elevating cyclic adenosine monophosphate (cAMP) levels, have been shown to promote anti-tumor immunity in various preclinical cancer models to enhance the efficacy of standard cancer treatments and other immune checkpoint blockade therapy. As a result, the CD39-CD73-adenosinergic pathway is now considered as one promising target in the field of cancer immunotherapy.

Extracellular ATP and adenosine levels are regulated by a complex system of enzymes, transporters and receptors. The canonical pathway leading to extracellular adenosine production involves the degradation of extracellular ATP (either actively secreted via pannexin channels or passively accumulating as a result of cell death) by the action membrane-bound ecto-nucleotidases, notably the rate-limiting enzymes CD39 and CD73. Extracellular ATP is first hydrolyzed by CD39, which catalyzes its degradation into ADP and AMP, then CD73 captures hydrolyzed extracellular AMP to adenosine. CD73-derived adenosine has a very short half-life in physiological conditions (few seconds). It is then either catabolized to inosine by membrane-bound adenosine deaminase, transported by equilibrative (ENTs) and/or concentrative (CNTs) nucleoside transporters, or used by specific adenosine receptors in an autocrine or paracrine manner.

Extracellular adenosine plays a critical endogenous distress signal in response to various kinds of insults including hypoxia, nutrient deprivation, inflammation and tumorigenesis. In these situations, accumulation of extracellular adenosine and stimulation of adenosine receptors (predominantly A2a and A2b subtypes) protect organs against injuries and excessive inflammation, and promote tissue repair. Limitation of vascular leakage, stimulation of angiogenesis, activation of extracellular matrix remodeling and suppression of immune cell activation, are the main mechanisms by which extracellular adenosine exerts its tissue-protecting functions. However, such adenosine-mediated defense mechanisms can also contribute to the development and progression of pathological conditions, including tumorigenesis.

Tumors co-opt the activities of the purinergic CD39/CD7³/adenosine system to shape the immune landscape in the tumor microenvironment at multiple levels. For example, tumor cells and tumor-associated Treg use CD73-dependent adenosine generation to dampen intratumoral immune responses, particularly in hypoxic tumors. The re-direction of the immune response involved suppression of T cell effector functions through CD73-dependent production of extracellular adenosine by CD39⁺/CD73⁺ Treg and signaling via stimulation of the ADORA2A on effector T cells. Adenosine and ADORA2A thus participate in shaping an immunosuppressive tumor microenvironment by negatively regulating CD8+ T cells. An adenosine-dependent suppression of immunosurveillance via IFN-γ, NK cells, and CD8+ T cells had also been demonstrated in other pre-clinical models. Finally, the creation of an immunosuppressive tumor microenvironment involved the expansion of immunosuppressive myeloid cells, e.g., myeloid-derived suppressor cells, M2-like macrophages, and potentially N2-like neutrophils.

In addition, the CD73/adenosine system also supports tumor growth-promoting neovascularization and tumor metastasis. Chemotherapy resistance though part of these actions can also be attributed to the CD73/adenosine-induced modulation of immune cell types in the tumor microenvironment. Thus, CD73 antibodies can be potentially used as cancer therapies.

Experimental animal models are an indispensable research tool for studying the effects of these antibodies (e.g., CD73 antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U. S. Pat. No. 4, 683, 195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wuetal. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### NT5E (CD73)

CD73, or Ecto-5′-nucleotidase (also known as NT5E or 5′-ribonucleotide phosphohydrolase), is encoded by the NT5E gene. The CD73 protein catalyzes the conversion at neutral pH of purine 5-prime mononucleotides to nucleosides. The preferred substrate for CD73 is adenosine monophosphate (AMP). The enzyme has a dimer of 2 identical 70-kD subunits bound by a glycosyl phosphatidyl inositol linkage to the external face of the plasma membrane. The enzyme is often used as a marker of lymphocyte differentiation. A deficiency of CD73 occurs in a variety of immunodeficiency diseases. CD73 is expressed to variable extents in different tissues, and is specially abundant in the colon, kidney, brain, liver, heart, lung, spleen, lymph nodes and the bone marrow. In the vasculature, CD73 is predominantly associated with the endothelium of vessels such as the aorta, carotid and coronary artery, as well as on afferent lymphatic endothelium, where it participates in the regulation of leukocyte trafficking. In the immune system, CD73 is found on the surface of macrophages, lymphocytes, regulatory T cells (Treg) and dendritic cells.

There are two known transcript variants that encode two different CD73 isoforms. Variant 1 represents the longer transcript and encodes the longer isoform 1, with 9 exons and a translation length of 574 residues (NP_002517.1; SEQ ID NO: 2). Variant 2 lacks an alternate in-frame exon compared to variant 1, consists of 8 exons and has a translation length of 524 residues (NP_001191742.1; SEQ ID NO: 31).

Recent work suggests that CD73 participates in the process of tumor immunoescape by inhibiting the activation, clonal expansion and homing of tumor-specific T cells, and thus impairing tumor cell killing by cytolytic effector T lymphocytes, dictating a substantial component of the suppressive capabilities of Treg and Th17 cells, and enhancing the conversion of anti-tumor type 1 macrophages into pro-tumor type 2 macrophage. Particularly, CD73, facilitating the pericellular generation of adenosine, is responsible for a substantial component of the immunosuppressive and anti-inflammatory functions of Treg cells. The immunosuppressive action of Treg-derived adenosine can be ascribed to the activation of A2A receptors expressed on T effector cells. In addition, adenosine triggers a self-reinforcing autocrine loop of Treg function, as the stimulation of A2A receptors on Tregs elicits cell expansion and increases their immunoregulatory activity. In parallel, A2A receptor activation on effector cells inhibits T-cell—mediated cytotoxicity and causes a reduction of cytokine production and T-cell proliferation. CD73-derived adenosine, produced by Tregs, inhibits NF-κB activation in effector T cells through A2A receptors, thereby reducing the release of a broad range of pro-inflammatory cytokines and chemokines. Thus, targeting CD73 with small molecule inhibitors or monoclonal antibodies can provide tumor inhibitory effects.

Pharmacological blockade of CD73 can also synergize with other currently available antineoplastic agents, such as anthracycline, anti-cytotoxic T-lymphocyte antigen (CTLA)-4 antibody and anti-programmed cell death protein (PD)-1 antibody.

A detailed description of CD73 and its function can be found, e.g., in Hammami, Akil, et al. “Targeting the adenosine pathway for cancer immunotherapy.” Seminars in immunology. Vol. 42. Academic Press, 2019; de Leve, Simone, Florian Wirsdorfer, and Verena Jendrossek. “Targeting the immunomodulatory CD73/adenosine system to improve the therapeutic gain of radiotherapy.” Frontiers in immunology 10 (2019): 698; Antonioli et al. “Anti-CD73 in cancer immunotherapy: awakening new opportunities.” Trends in cancer 2.2 (2016): 95-109; each of which is incorporated by reference in its entirety.

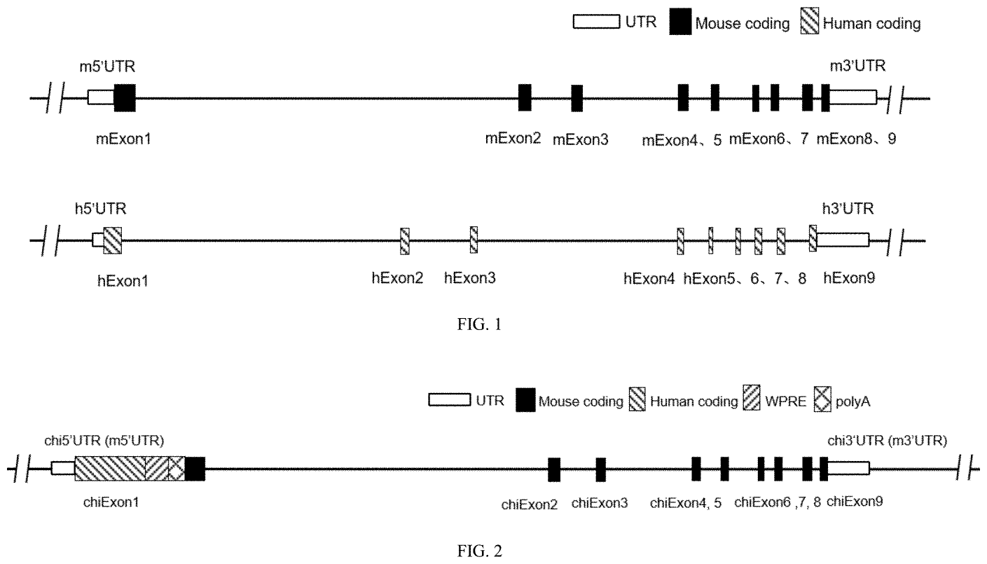

In human genomes, NT5E gene (Gene ID: 4907) locus has nine exons, exon 1, exon 2, exon 3, exon 4, exon 5, exon 6, exon 7, exon 8, and exon 9 (FIG. 1). A signal peptide is located in exon 1 of the human NT5E gene. The nucleotide sequence for human CD73 mRNA is NM_002526.3 (SEQ ID NO: 3), and the amino acid sequence for human CD73 is NP_002517.1 (SEQ ID NO: 4). The location for each exon and each region in human NT5E nucleotide sequence and CD73 amino acid sequence is listed below:

TABLE 1

| Human NT5E | NM_002526.3 | NP_002517.1 |
|---|---|---|
| (approximate location) | 4086bp | 574aa |
| Exon 1 | 1-895 | 1-113 |
| Exon 2 | 896-1118 | 114-187 |
| Exon 3 | 1119-1307 | 188-250 |
| Exon 4 | 1308-1505 | 251-316 |
| Exon 5 | 1506-1660 | 317-368 |
| Exon 6 | 1661-1766 | 369-403 |
| Exon 7 | 1767-1916 | 404-453 |
| Exon 8 | 1917-2117 | 454-520 |
| Exon 9 | 2118-4068 | 521-574 |
| Signal peptide | 557-634 | 1-26 |
| Donor region in Example | 557-2281 | 1-574 |

In mice, NT5E gene (Gene ID: 23959) locus has nine exons, exon 1, exon 2, exon 3, exon 4, exon 5, exon 6, exon 7, exon 8, and exon 9 (FIG. 1). A signal peptide is located in exon 1 of the mouse NT5E gene. The nucleotide sequence for mouse CD73 mDNA is NM_011851.4 (SEQ ID NO: 1), the amino acid sequence for mouse CD73 is NP_035981.1 (SEQ ID NO: 2). The location for each exon and each region in the mouse NT5E nucleotide sequence and CD73 amino acid sequence is listed below:

TABLE 2

| Mouse NT5E | NM_011851.4 | NP_035981.1 |
|---|---|---|
| (approximate location) | 3580 bp | 576 aa |
| Exon 1 | 1-422 | 1-115 |
| Exon 2 | 423-645 | 116-189 |
| Exon 3 | 646-834 | 190-252 |
| Exon 4 | 835-1032 | 253-318 |
| Exon 5 | 1033-1187 | 319-370 |
| Exon 6 | 1188-1293 | 371-405 |
| Exon 7 | 1294-1443 | 406-455 |
| Exon 8 | 1444-1644 | 456-522 |
| Exon 9 | 1645-3580 | 523-576 |
| Signal peptide | 78-161 | 1-28 |

The mouse NT5E gene (Gene ID: 23959) located in Chromosome 9 of the mouse genome, which is located from 88327609 to 88372089 of NC 000075.6 (GRCm38.p4 (GCF_000001635.24). The 5′-UTR is from 88,327,197 to 53618607, exon 1 is from 88,327,197 to 88,328,030, the first intron is from 88,328,031 to 88,352,262, exon 2 is from 88,352,263 to 88,352,485, the second intron is from 88,352,486 to 88,355,586, exon 3 is from 88,355,587 to 88,355,775, the third intron is from 88,355,776 to 88,363,435, exon 4 is from 88,363,436 to 88,363,633, the fourth intron is from 88,363,634 to 88,364,667, exon 5 is from 88,364,668 to 88,364,822, the fifth intron is from 88,364,823 to 88,366,361, exon 6 is from 88,366,362 to 88,366,467, the sixth intron is from 88,366,468 to 88,367,230, exon 7 is from 88,367,230 to 88,367,380, the seventh intron is from 88,367,381 to 88,369,045, exon 8 is from 88,369,046 to 88,369,246, the eighth intron is from 88,369,247 to 88,370,153, exon 9 is from 88,370,154 to 88,372,092, and the 3′-UTR is from 88,370,318 to 88,372,092, based on transcript NM_011851.4. All relevant information for mouse NT5E locus can be found in the NCBI website with Gene ID: 23959, which is incorporated by reference herein in its entirety.

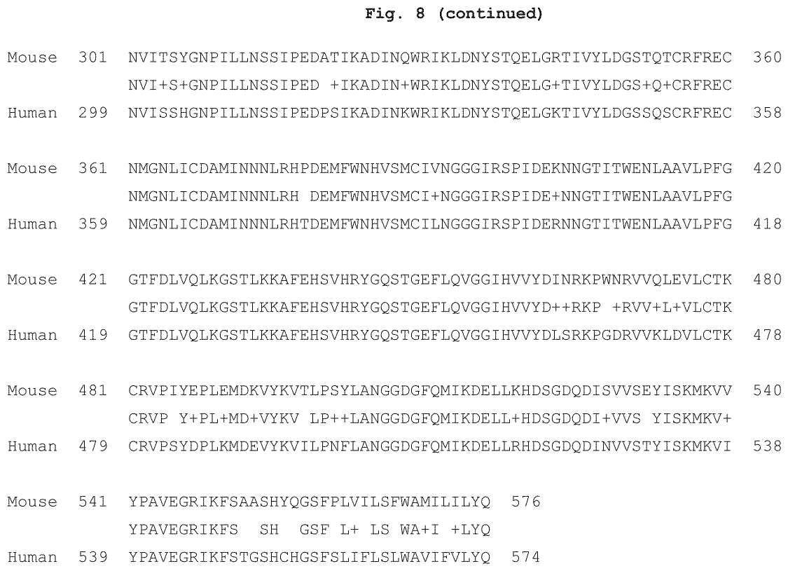

FIG. 8 shows the alignment between mouse CD73 amino acid sequence (NP_035981.1; SEQ ID NO: 2) and human CD73 amino acid sequence (NP_002517.1; SEQ ID NO: 4). Thus, the corresponding amino acid residue or region between human and mouse CD73 can be found in FIG. 8.

CD73 genes, proteins, and locus of the other species are also known in the art. For example, the gene ID for CD73 in Rattus norvegicus (Norway rat) is 58813, the gene ID for CD73 in Macaca mulatta (Rhesus monkey) is 696509, the gene ID for CD73 in Canis lupus familiaris (dog) is 474984, and the gene ID for CD73 in Felis catus (domestic cat) is 101087744. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) CD73 nucleotide sequence and/or amino acid sequences.

In some embodiments, a sequence encoding a human CD73 (SEQ ID NO: 4 or SEQ ID NO: 31) or a chimeric CD73 is inserted to the mouse CD73 locus.

In some embodiments, the entire sequence of human or chimeric exon 1, exon 2, exon 3, exon 4, exon 5, exon 6, exon 7, exon 8, exon 9, and/or signal peptide, is inserted to the mouse endogenous locus (e.g., within exon 1, immediately before the translation start codon). In some embodiments, a “region” or “portion” of human or chimeric exon 1, exon 2, exon 3, exon 4, exon 5, exon 6, exon 7, exon 8, exon 9, and/or signal peptide, is inserted to the mouse endogenous locus. In some embodiments, at least 10% 20%, 30%, 40%, 50% (e.g., 20%) of the sequence is identical (e.g., continuously) to the human CD73 sequence or part thereof (e.g., exon 1, exon 2, exon 3, exon 4, exon 5, exon 6, exon 7, exon 8, exon 9), and at least 10% 20%, 30%, 40%, 50% (e.g., 20%) of the sequence is identical (e.g., continuously) to the mouse CD73 sequence or part thereof (e.g., exon 1, exon 2, exon 3, exon 4, exon 5, exon 6, exon 7, exon 8, exon 9).

In some embodiments, the entire sequence of mouse exon 1, exon 2, exon 3, exon 4, exon 5, exon 6, exon 7, exon 8, exon 9, and/or signal peptide, are replaced by the corresponding human sequence. In some embodiments, a “region” or “portion” of mouse exon 1, exon 2, exon 3, exon 4, exon 5, exon 6, exon 7, exon 8, exon 9, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, or signal peptide. In some embodiments, a region, a portion, or the entire sequence of mouse exon 1, exon 2, exon 3, exon 4, exon 5, exon 6, exon 7, exon 8, and/or exon 9 (e.g., exon 2, exon 3, exon 4, exon 5, exon 6, exon 7, exon 8) are replaced by the human exon 1, exon 2, exon 3, exon 4, exon 5, exon 6, exon 7, exon 8, and/or exon 9 (e.g., exon 2, exon 3, exon 4, exon 5, exon 6, exon 7, exon 8) sequence.

In some embodiments, the present disclosure also provides a chimeric (e.g., humanized) CD73 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD73 mRNA sequence (e.g., SEQ ID NO: 1), mouse CD73 amino acid sequence (e.g., SEQ ID NO: 2), or a portion thereof (e.g., exon 1, exon 2, exon 3, exon 4, exon 5, exon 6, exon 7, exon 8, and/or exon 9); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD73 mRNA sequence (e.g., SEQ ID NO: 3), human CD73 amino acid sequence (e.g., SEQ ID NO: 4, SEQ ID NO: 31), or a portion thereof (e.g., exon 1, exon 2, exon 3, exon 4, exon 5, exon 6, exon 7, exon 8, and/or exon 9).

In some embodiments, the nucleic acids as described herein are operably linked to an endogenous mouse CD73 promotor (e.g., at endogenous mouse CD73 transcription start site) or the 5′-UTR of the endogenous CD73 gene. In some embodiments, the nucleic acids is contiguously linked to 5′-UTR of the endogenous CD73 gene. In some embodiments, the nucleic acid is inserted before (e.g., immediately before the translation start codon of the endogenous CD73 gene).

In some embodiments, the nucleic acids as described herein are operably linked to a Woodchuck Hepatitis Virus (WHP) Posttranscriptional Regulatory Element (WPRE) and/or a polyA (polyadenylation) signal sequence. The WPRE element is a DNA sequence that, when transcribed, creates a tertiary structure enhancing expression. The sequence can be used to increase expression of genes delivered by viral vectors. WPRE is a tripartite regulatory element with gamma, alpha, and beta components. The sequence for the alpha component is shown below:

(SEQ ID NO: 30)
GCCACGGCGGAACTCATCGCCGCCTGCCTTGCCCGCTGCTGGACAGGGGC
TCGGCTGTTGGGCACTGACAATTCCGTGGT

When used alone without the gamma and beta WPRE components, the alpha component is only 9% as active as the full tripartite WPRE. The full tripartite WPRE sequence is set forth in SEQ ID NO: 8. In some embodiments, the WPRE sequence has a sequence that is at least 70%, 80%, 90%, or 95% identical to SEQ ID NO: 8.

In some embodiments, the polyA (polyadenylation) signal sequence has a sequence that is at least 70%, 80%, 90%, or 95% identical to SEQ ID NO: 9.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD73 nucleotide sequence (e.g., exon 1, exon 2, exon 3, exon 4, exon 5, exon 6, exon 7, exon 8, exon 9 or NM_011851.4 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD73 nucleotide sequence (e.g., exon 1, exon 2, exon 3, exon 4, exon 5, exon 6, exon 7, exon 8, exon 9 or NM_011851.4 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD73 nucleotide sequence (e.g., exon 1, exon 2, exon 3, exon 4, exon 5, exon 6, exon 7, exon 8, exon 9, or NM_002526.3 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD73 nucleotide sequence (e.g., exon 1, exon 2, exon 3, exon 4, exon 5, exon 6, exon 7, exon 8, exon 9 or NM_002526.3 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD73 amino acid sequence (e.g., exon 1, exon 2, exon 3, exon 4, exon 5, exon 6, exon 7, exon 8, exon 9 or NP_035981.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD73 amino acid sequence (e.g., exon 1, exon 2, exon 3, exon 4, exon 5, exon 6, exon 7, exon 8, exon 9 or NP_035981.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD73 amino acid sequence (e.g., exon 1, exon 2, exon 3, exon 4, exon 5, exon 6, exon 7, exon 8, exon 9, NP_002517.1 (SEQ ID NO: 4) or NP_001191742.1 (SEQ ID NO: 31)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD73 amino acid sequence (e.g., exon 1, exon 2, exon 3, exon 4, exon 5, exon 6, exon 7, exon 8, exon 9, NP_002517.1 (SEQ ID NO: 4), or NP_001191742.1 (SEQ ID NO: 31)).

The present disclosure also provides a humanized CD73 mouse amino acid sequence, wherein the amino acid sequence is selected from the group consisting of:

a) an amino acid sequence shown in SEQ ID NO: 4 or SEQ ID NO: 31;

b) an amino acid sequence having a homology of at least 90% with or at least 90% identical to the amino acid sequence shown in SEQ ID NO: 4 or SEQ ID NO: 31;

c) an amino acid sequence encoded by a nucleic acid sequence, wherein the nucleic acid sequence is able to hybridize to a nucleotide sequence encoding the amino acid shown in SEQ ID NO: 4 or SEQ ID NO: 31 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 4 or SEQ ID NO: 31;

e) an amino acid sequence that is different from the amino acid sequence shown in SEQ ID NO: 4 or SEQ ID NO: 31 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 4 or SEQ ID NO: 31.

The present disclosure also relates to a CD73 nucleic acid (e.g., DNA or RNA) sequence, wherein the nucleic acid sequence can be selected from the group consisting of:

a) a nucleic acid sequence as shown in SEQ ID NO: 3, SEQ ID NO: 7, SEQ ID NO: 29, or a nucleic acid sequence encoding a homologous CD73 amino acid sequence of a humanized mouse;

b) a nucleic acid sequence that is shown in SEQ ID NO: 3, SEQ ID NO: 7, or SEQ ID NO: 29;

c) a nucleic acid sequence that is able to hybridize to the nucleotide sequence as shown in SEQ ID NO: 3, SEQ ID NO: 7, or SEQ ID NO: 29 under a low stringency condition or a strict stringency condition;

d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, SEQ ID NO: 7, or SEQ ID NO: 29;

e) a nucleic acid sequence that encodes an amino acid sequence, wherein the amino acid sequence has a homology of at least 90% with or at least 90% identical to the amino acid sequence shown in SEQ ID NO: 3, SEQ ID NO: 7, or SEQ ID NO: 29;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 3, SEQ ID NO: 7, or SEQ ID NO: 29;

g) a nucleic acid sequence that encodes an amino acid sequence, wherein the amino acid sequence is different from the amino acid sequence shown in SEQ ID NO: 3, SEQ ID NO: 7, or SEQ ID NO: 29 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 3, SEQ ID NO: 7, or SEQ ID NO: 29.

The present disclosure further relates to a CD73 genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 3, SEQ ID NO: 7, or SEQ ID NO: 29.

The disclosure also provides an amino acid sequence that has a homology of at least 90% with, or at least 90% identical to the sequence shown in SEQ ID NO: 4 or SEQ ID NO: 31, and has protein activity. In some embodiments, the homology with the sequence shown in SEQ ID NO: 4 or SEQ ID NO: 31 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 4 or SEQ ID NO: 31 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 3, SEQ ID NO: 7, or SEQ ID NO: 29, and encodes a polypeptide that has protein activity. In some embodiments, the homology with the sequence shown in SEQ ID NO: 3, SEQ ID NO: 7, or SEQ ID NO: 29 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 3, SEQ ID NO: 7, or SEQ ID NO: 29 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CD73 from an endogenous non-human CD73 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CD73 locus that comprises an exogenous sequence (e.g., a human sequence), e.g., an insertion of an exogenous sequence or a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized CD73 gene or a humanized CD73 nucleic acid. In some embodiments, at least one or more portions of the gene or the nucleic acid is from the human CD73 gene, at least one or more portions of the gene or the nucleic acid is from a non-human CD73 gene. In some embodiments, the gene or the nucleic acid comprises a sequence that encodes a CD73 protein. The encoded CD73 protein is functional or has at least one activity of the human CD73 protein or the non-human CD73 protein, e.g., binding with AMP; hydrolyzing extracellular AMP; dampening intratumoral immune responses; suppression of T cell effector functions; negatively regulating of CD8⁺ T cells; suppression of immunosurveillance via IFN-γ, NK cells, and CD8⁺ T cells; expansion of immunosuppressive myeloid cells; supporting tumor growth-promoting neovascularization, tumor metastasis, and chemotherapy resistance; contributing to the development and progression of pathological conditions, including tumorigenesis; and/or limitation of vascular leakage, stimulation of angiogenesis, activation of extracellular matrix remodeling and suppression of immune cell activation.

In some embodiments, the chimeric protein or the chimeric polypeptide is a humanized CD73 protein or a humanized CD73 polypeptide. In some embodiments, at least one or more portions of the amino acid sequence of the protein or the polypeptide is from a human CD73 protein, and/or at least one or more portions of the amino acid sequence of the protein or the polypeptide is from a non-human CD73 protein. The humanized CD73 protein or the humanized CD73 polypeptide is functional or has at least one activity of the human CD73 protein or the non-human CD73 protein.

In some embodiments, the genetically modified non-human animal expresses a human CD73 protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized CD73 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of the entire or at least a portion of an endogenous non-human CD73 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include an insertion of a human or a chimeric CD73 sequence or a replacement of all or part of mature CD73 coding sequence with human mature CD73 coding sequence.

Genetically modified non-human animals that comprise a modification of an endogenous non-human CD73 locus. In some embodiments, the modification can comprise a human nucleic acid sequence encoding at least a portion of a mature CD73 protein (e.g., at least 10%, 20%, 30%, 40%, 50%, 60%, 70%, 80%, 90%, 95%, 96%, 97%, 98%, 99% or 100% identical to the human CD73 protein sequence). Although genetically modified cells are also provided that can comprise the modifications described herein (e.g., ES cells, somatic cells), in many embodiments, the genetically modified non-human animals comprise the modification of the endogenous CD73 locus in the germline of the animal.

Genetically modified animals can express a human CD73 and/or a chimeric (e.g., humanized) CD73 from endogenous mouse loci, wherein the endogenous mouse CD73 gene has been replaced with a human CD73 gene and/or a nucleotide sequence that encodes a region of human CD73 sequence or an amino acid sequence that is at least 10%, 20%, 30%, 40%, 50%, 60%, 70%, 80%, 90%, 95%, 96%, 97%, 98%, or 99% identical to the human CD73 sequence. In various embodiments, an endogenous non-human CD73 locus is modified in whole or in part to comprise human nucleic acid sequence encoding at least one protein-coding sequence of a mature CD73 protein.

In some embodiments, the genetically modified mice express the human CD73 and/or chimeric CD73 (e.g., humanized CD73) from endogenous loci that are under control of a mouse promoter. The human CD73 or the chimeric CD73 (e.g., humanized CD73) expressed in animal can maintain one or more functions of the wild-type mouse or human CD73 in the animal. For example, expressed CD73 can hydrolyze extracellular AMP, downregulate immune response, e.g., downregulate immune response by at least 10%, 20%, 30%, 40%, or 50%. Furthermore, in some embodiments, the animal does not express endogenous CD73. As used herein, the term “endogenous CD73” refers to CD73 protein that is expressed from an endogenous CD73 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human CD73 (NP_002517.1) (SEQ ID NO: 4) or NP_001191742.1 (SEQ ID NO: 31).

The genome of the genetically modified animal can comprise a replacement at an endogenous CD73 gene locus of a sequence encoding a region of endogenous CD73 with a sequence encoding a corresponding region of human CD73. In some embodiments, the sequence that is replaced is any sequence within the endogenous CD73 gene locus, e.g., exon 1, exon 2, exon 3, exon 4, exon 5, exon 6, exon 7, exon 8, exon 9, 5′-UTR, 3′-UTR, the first intron, the second intron, and the third intron, the fourth intron, the fifth intron, the sixth intron, the seventh intron, the eighth intron etc. In some embodiments, the sequence that is replaced is exon 1, exon 2, exon 3, exon 4, exon 5, exon 6, exon 7, exon 8, exon 9, or part thereof, of an endogenous mouse CD73 gene locus.

Because human CD73 and non-human CD73 (e.g., mouse CD73) sequences, in many cases, are different, antibodies that bind to human CD73 will not necessarily have the same binding affinity with non-human CD73 or have the same effects to non-human CD73. Therefore, the genetically modified animal having a human or a humanized extracellular region can be used to better evaluate the effects of anti-human CD73 antibodies in an animal model. In some embodiments, the genome of the genetically modified animal comprises a sequence encoding the extracellular region of human CD73.

In some embodiments, the non-human animal can have, at an endogenous CD73 gene locus, a nucleotide sequence encoding a chimeric human/non-human CD73 polypeptide, and wherein the animal expresses a functional CD73 on a surface of a cell of the animal. The human portion of the chimeric human/non-human CD73 polypeptide can comprise a portion of exon 1, exon 2, exon 3, exon 4, exon 5, exon 6, exon 7, exon 8, and/or exon 9 of human CD73. In some embodiments, the human portion of the chimeric human/non-human CD73 polypeptide can comprise a sequence that is at least 80%, 85%, 90%, 95%, or 99% identical to SEQ ID NO: 4 or SEQ ID NO: 31. Furthermore, the genetically modified animal can be heterozygous with respect to the modification (e.g., insertion or replacement) at the endogenous CD73 locus, or homozygous with respect to the modification at the endogenous CD73 locus.

In some embodiments, the humanized CD73 locus lacks a human CD73 5′-UTR. In some embodiment, the humanized CD73 locus comprises a rodent (e.g., mouse) 5′-UTR.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CD73 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CD73 in the genome of the animal.

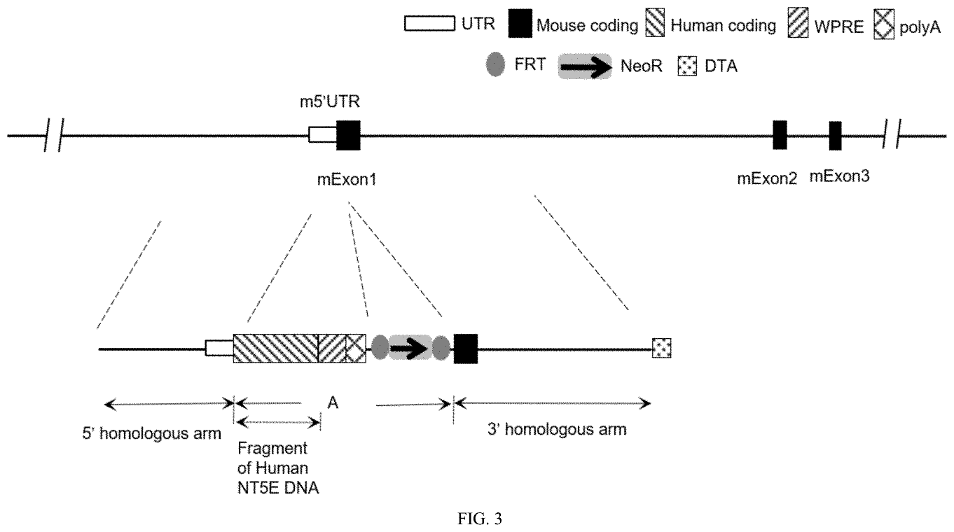

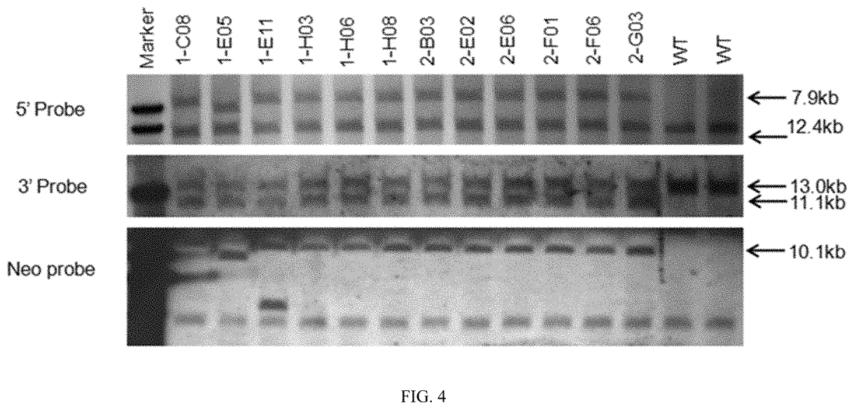

In some embodiments, the non-human mammal comprises the genetic construct as described herein (e.g., gene construct as shown in FIG. 2 or FIG. 3). In some embodiments, a non-human mammal expressing human or humanized CD73 is provided. In some embodiments, the tissue-specific expression of human or humanized CD73 protein is provided.

In some embodiments, the expression of human or humanized CD73 in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human CD73 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized CD73 protein.

### Vectors

The present disclosure relates to a targeting vector, comprising: a) a DNA fragment homologous to the 5′ end of a region to be altered (5′ arm), which is selected from the CD73 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CD73 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000075.6; c) the DNA fragment homologous to the 3′ end of the region to be altered (3′ arm) is selected from the nucleotide sequences that have at least 90% homology to the NCBI accession number NC_000075.6.

In some embodiments, a) the DNA fragment homologous to the 5′ end of a region to be altered (5′ arm) is selected from the nucleotides from the position 88324697 to the position 88327685 of the NCBI accession number NC_000075.6; c) the DNA fragment homologous to the 3′ end of the region to be altered (3′ arm) is selected from the nucleotides from the position 88327704 to the position 88332552 of the NCBI accession number NC_000075.6.

In some embodiments, the length of the selected genomic nucleotide sequence in the targeting vector can be more than about 1 kb, about 2 kb, about 3 kb, about 3.5 kb, about 4 kb, about 4.5 kb, about 5 kb, about 5.5 kb, or about 6 kb.

In some embodiments, the region to be inserted is exon 1, exon 2, exon 3, exon 4, exon 5, exon 6, exon 7, exon8, and/or exon 9 of human CD73 gene. In some embodiments, the region to be altered is exon 1, exon 2, exon 3, exon 4, exon 5, exon 6, exon 7, exon8, and/or exon 9 of CD73 gene.

The targeting vector can further include a selected gene marker.

In some embodiments, the sequence of the 5′ arm is shown in SEQ ID NO: 5; and the sequence of the 3′ arm is shown in SEQ ID NO: 6.

In some embodiments, the sequence is derived from human (e.g., 557-2281 of NM_002526.3). For example, the target region in the targeting vector is a part or entirety of the nucleotide sequence of a human CD73, preferably exon 1, exon 2, exon 3, exon 4, exon 5, exon 6, exon 7, exon 8 and/or exon 9 of the human CD73. In some embodiments, the nucleotide sequence of the humanized CD73 encodes the entire or the part of human CD73 protein with the NCBI accession number NP_002517.1 (SEQ ID NO: 4) or NP_001191742.1 (SEQ ID NO: 31).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., non-homologous end-joining (NHEJ), homologous recombination (HR), zinc finger nucleases (ZFNs), transcription activator-like effector-based nucleases (TALEN), homing endonuclease (megakable base ribozyme), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides inserting in at least one cell of the animal, at an endogenous CD73 gene locus, a sequence encoding a human or chimeric CD73. In some embodiments, the disclosure provides replacing in at least one cell of the animal, at an endogenous CD73 gene locus, a sequence encoding a region of an endogenous CD73 with a sequence encoding a corresponding region of human or chimeric CD73. In some embodiments, the modification occurs in a germ cell, a somatic cell, a blastocyst, or a fibroblast, etc. The nucleus of a somatic cell or the fibroblast can be inserted into an enucleated oocyte.

FIG. 3 shows a humanization strategy for a mouse CD73 locus. In FIG. 3, the targeting strategy involves a vector comprising the 5′ end homologous arm, human CD73 gene fragment, helper sequences (including WPRE, polyA, Neo cassette), and 3′ homologous arm. The process can involve replacing endogenous CD73 sequence with human sequence by homologous recombination. In some embodiments, the cleavage at the upstream and the downstream of the target site (e.g., by zinc finger nucleases, TALEN or CRISPR) can result in DNA double strands break, and the homologous recombination can be used to replace endogenous CD73 sequence with human CD73 sequence.

Thus, in some embodiments, the methods for making a genetically modified, humanized animal, can include the step of replacing at an endogenous CD73 locus (or site), a nucleic acid encoding a sequence encoding a region of endogenous CD73 with a sequence encoding a human or chimeric CD73. The sequence can include a region (e.g., a part or the entire region) of exon 1, exon 2, exon 3, exon 4, exon 5, exon 6, exon 7, exon 8, and/or exon 9 of a human CD73 gene.

In some embodiments, the methods of modifying a CD73 locus of a mouse to express a chimeric human/mouse CD73 peptide can include the steps of replacing at the endogenous mouse CD73 locus a nucleotide sequence encoding a mouse CD73 with a nucleotide sequence encoding a human CD73, thereby generating a sequence encoding a chimeric human/mouse CD73.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a CD73 gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Genetically modified animals that express human or humanized CD73 protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CD73, which are useful for testing agents that can decrease or block the interaction between CD73 and CD73 substrates (e.g., AMP), testing whether an agent can increase or decrease the immune response, and/or determining whether an agent is an CD73 inhibitor, agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of an anti-CD73 antibody for the treatment of cancer. The methods involve administering the anti-CD73 antibody (e.g., anti-human CD73 antibody) to the animal as described herein, wherein the animal has a tumor; and determining the inhibitory effects of the anti-CD73 antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the anti-CD73 antibody prevents AMP from binding to CD73. In some embodiments, the anti-CD73 antibody does not prevent AMP from binding to CD73.

In some embodiments, the genetically modified animals can be used for determining whether an anti-CD73 antibody is a CD73 inhibitor, agonist or antagonist. In some embodiments, the methods as described herein are also designed to determine the effects of the agent (e.g., anti-CD73 antibodies) on CD73, e.g., whether the agent can stimulate immune cells or inhibit immune cells (e.g., macrophages, B cells, or DC), whether the agent can increase or decrease the production of cytokines, whether the agent can activate or deactivate immune cells (e.g., macrophages, B cells, or DC), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGIrv). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-CD73 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-CD73 antibody is designed for treating solid tumors, glioma, head and neck cancer, melanoma, thyroid cancer, breast cancer, pancreatic cancer, colon cancer, bladder cancer, ovarian cancer, prostate cancer, or leukemia.

In some embodiments, the anti-CD73 antibody is designed for treating various autoimmune diseases. Thus, the methods as described herein can be used to determine the effectiveness of an anti-CD73 antibody in inhibiting immune response.

The present disclosure also provides methods of determining toxicity of an antibody (e.g., anti-CD73 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CD73 gene function, human CD73 antibodies, drugs for human CD73 targeting sites, the drugs or efficacies for human CD73 targeting sites, the drugs for immune-related diseases and antitumor drugs.

Genetically Modified Animal Model with Two or More Human or Chimeric Genes

The present disclosure further relates to methods for generating genetically modified animal model with two or more human or chimeric genes. The animal can comprise a human or chimeric CD73 gene and a sequence encoding an additional human or chimeric protein.

In some embodiments, the additional human or chimeric protein can be programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (TNFRSF4 or OX40).

The methods of generating genetically modified animal model with two or more human or chimeric genes (e.g., humanized genes) can include the following steps:

(a) using the methods of introducing human CD73 gene or chimeric CD73 gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, CTLA-4, LAG-3, BTLA, PD-L1, CD3, CD27, CD28, CD47, CD137, CD154, TIGIT, TIM-3, GITR, SIRPα, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the CD73 humanization is directly performed on a genetically modified animal having a human or chimeric PD-1, CTLA-4, BTLA, PD-L1, CD3, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CD73 antibody and an additional therapeutic agent for the treatment of cancer. The methods include administering the anti-CD73 antibody and the additional therapeutic agent to the animal, wherein the animal has a tumor; and determining the inhibitory effects of the combined treatment to the tumor. In some embodiments, the additional therapeutic agent is an antibody that specifically binds to PD-1, CTLA-4, BTLA, PD-L1, CD3, CD27, CD28, CD47, CD137, CD154, TIGIT, TIM-3, GITR, SIRPα, or OX40. In some embodiments, the additional therapeutic agent is an anti-CTLA4 antibody (e.g., ipilimumab), an anti-PD-1 antibody (e.g., nivolumab), or anthracycline.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors).

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

C57BL/6 mice and Flp mice were purchased from the China Food and Drugs Research Institute National Rodent Experimental Animal Center.

Humanized PD-1 mice and humanized CTLA4 mice were obtained from Beijing Biocytogen Co., Ltd. (Catalog number: B-CM-001, B-CM-024, respectively).

AIO kit was obtained from Beijing Biocytogen Co., Ltd. (Catalog number: BCG-DX-004).

APC anti-mouse CD73 antibody (mCD73 APC) was purchased from BioLegend, Inc. (Catalog number: 127209).

PerCP/Cy55 anti-mouse TCR beta chain antibody (mTcRβ PerCP) was purchased from BioLegend, Inc. (Catalog number 109228).

PE anti-human CD73 (Ecto-5′-nucleotidase) antibody (hCD73 PE) was purchased from BioLegend, Inc. (Catalog number 344003).

EcoRV, NdeI, SacI restriction enzymes were purchased from NEB (Catalog numbers: R0195S, R3193M, R3156M).

### Example 1: NT5E (CD73) Gene Humanized Mice

A schematic diagram comparing the mouse NT5E gene (NCBI Gene ID: 23959, Primary source: MGI: 99782, UniProt ID: P07750; based on the transcript of NCBI accession number NM_011851.4 NP_035981.1, whose mRNA sequence is shown in SEQ ID NO: 1, and the corresponding protein sequence is shown in SEQ ID NO: 2) and the human NT5E gene (NCBI Gene ID: 4907, Primary source: HGNC: 8021, UniProt ID: P21589; based on the transcript of NCBI accession number NM_002526.3→NP_002517.1, whose mRNA sequence was shown in SEQ ID NO: 3, and the corresponding protein sequence is shown in SEQ ID NO: 4) are shown in FIG. 1.

Given that human or mouse NT5E has multiple isoforms or transcripts, the methods described herein can be applied to other isoforms or transcripts. In particular, human NT5E has another isoform or transcript, but this transcript lacks an in-frame exon and nuclease activity. In addition, it has a low expression level under normal conditions, so the NM_002526.3 transcript was selected for experiments.

In this experiment, a gene sequence encoding human CD73 protein was introduced into the endogenous mouse NT5E locus, such that the mouse can express human CD73 protein. Mouse cells were modified. The human CD73 protein coding sequence was inserted before the start codon (ATG) sequence in the endogenous mouse NT5E locus.

In order to increase the CD73 protein expression level, Woodchuck Hepatitis Virus (WHP) Posttranscriptional Regulatory Element (WPRE) and polyA (polyadenylation) signal sequence were added after the human CD73 coding sequence. The schematic diagram of the humanized mouse NT5E gene is shown in FIG. 2. The mouse regulates the expression of human NT5E sequence by an endogenous promoter, and the CD73 protein expressed in vivo is human CD73 protein. A targeting strategy was further designed as shown in FIG. 3. The mouse coding region on the humanized mouse NT5E gene shown in FIG. 2 will not be transcribed or translated, due to the presence of a stop codon and the poly-A signal after the inserted recombinant sequence.

Given that human NT5E or mouse NT5E has multiple isoforms or transcripts, the methods described herein can be applied to other isoforms or transcripts.

As the schematic diagram of the targeting strategy in FIG. 3 shows, the recombinant vector contained homologous arm sequences upstream and downstream of mouse NT5E (about 2989 bp upstream of the endogenous NT5E gene start codon ATG and 4849 bp downstream of the ATG codon), and a DNA fragment (abbreviated as A fragment) comprising the human NT5E sequence and the helper sequences WPRE and polyA. Wherein, the upstream homologous arm sequence (5′ homologous arm, SEQ ID NO: 5) is identical to the nucleotide sequence 88324697-88327685 of NCBI accession number NC_000075.6, and the downstream homologous arm sequence (3′ homologous arm, SEQ ID NO: 6) is identical to the nucleotide sequence 82327704-88332552 of NCBI accession number NC_000075.6; the human NT5E sequence (SEQ ID NO: 7) is identical to the nucleotide sequence 557-2281 of NCBI accession number NM_002526.3; the WPRE sequence is set forth in SEQ ID NO: 8, the polyA signal sequence is set forth in SEQ ID NO: 9; and the A fragment sequence is set forth in SEQ ID NO: 29.

The A fragment also included an antibiotic resistance gene for positive clone screening (neomycin phosphotransferase encoding sequence Neo), and two Frt recombination sites on both sides of the antibiotic resistance gene that formed a Neo cassette. The human NT5E sequence, WPRE, polyA and Neo cassette sequences were arranged in the 5′ to 3′ direction on the A fragment, and the sequence containing the human NT5E gene is directly linked to the upstream homologous arm sequence. The downstream junction of the Neo cassette and the mouse NT5E locus was designed to be within

(SEQ ID NO: 10)
5′-GAACTTCATCAGTCAGGTACATAATGGTGGATCCGATATCAAGGTAC
CCAAGTGGCTGCTTCTCGCACTGAGCGCTCTACTACCACAG-3′,

wherein the “C” of the sequence “ATATC” is the last nucleotide of the Neo cassette, and the first “A” in the sequence “AAGGT” is the first nucleotide of the mouse sequence.

In addition, a negative selection marker (a sequence encoding the diphtheria toxin A subunit (DTA)) was also inserted downstream of the 3′ homologous arm of the recombinant vector.

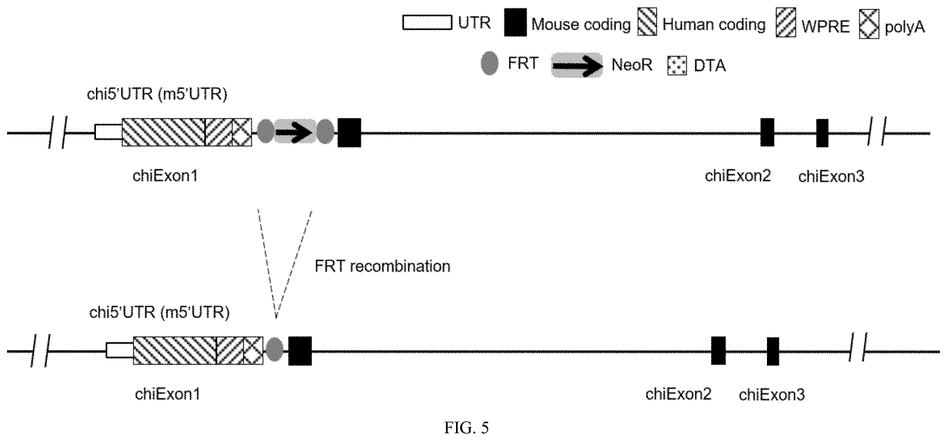

Vector construction can be carried out by restriction enzyme digestion and ligation. The constructed recombinant vector sequence can be initially verified by restriction enzyme digestion, followed by sequencing verification. The correct recombinant vector was electroporated and transfected into embryonic stem cells of C57BL/6 mice. The positive selectable marker gene was used to screen the cells, and the integration of exogenous genes was confirmed by PCR and Southern Blot. PCR and Southern Blot results (digested with ScaI or EcoRV or NdeI, respectively, and hybridized with 3 probes) for some of the clones were shown in FIG. 4. The test results showed that the 12 PCR-positive clones, except for 1-Ell, were identified as positive heterozygous clones and no random insertions were detected.

The PCR assay included the following primers:

(SEQ ID NO: 11)
F1: 5′ - GCTCGACTAGAGCTTGCGGA -3′,
(SEQ ID NO: 12)
R1: 5′ - TAGAGCCCCAGTTCAAAAGCAACCT -3′,
(SEQ ID NO: 13)
F2: 5′ - ACAAAGCAGGTGGACAGCATCCTAC-3′,
(SEQ ID NO: 14)
R2: 5′ - ATCTGCTGAACCTTGGTGAAGAGCC-3′;

Southern Blot assays included the following probes:

5′ Probe:
(SEQ ID NO: 15)
F: 5′ - CCTTCTTTTTCGGCGACCGAGC -3′,
(SEQ ID NO: 16)
R: 5′ - GCTGGCTAGAGCGCGTTGAGC -3′;
3′ Probe:
(SEQ ID NO: 17)
F: 5′ - GGAGGAAATGGAAGCAGGCCAGG -3′,
(SEQ ID NO: 18)
R: 5′ - CTAGCCAGTGTCACCCCCAAGG -3′;
Neo Probe:
(SEQ ID NO: 19)
F: 5′ - GGATCGGCCATTGAACAAGATGG -3′,
(SEQ ID NO: 20)
R: 5′ - CAGAAGAACTCGTCAAGAAGGCG-3′.

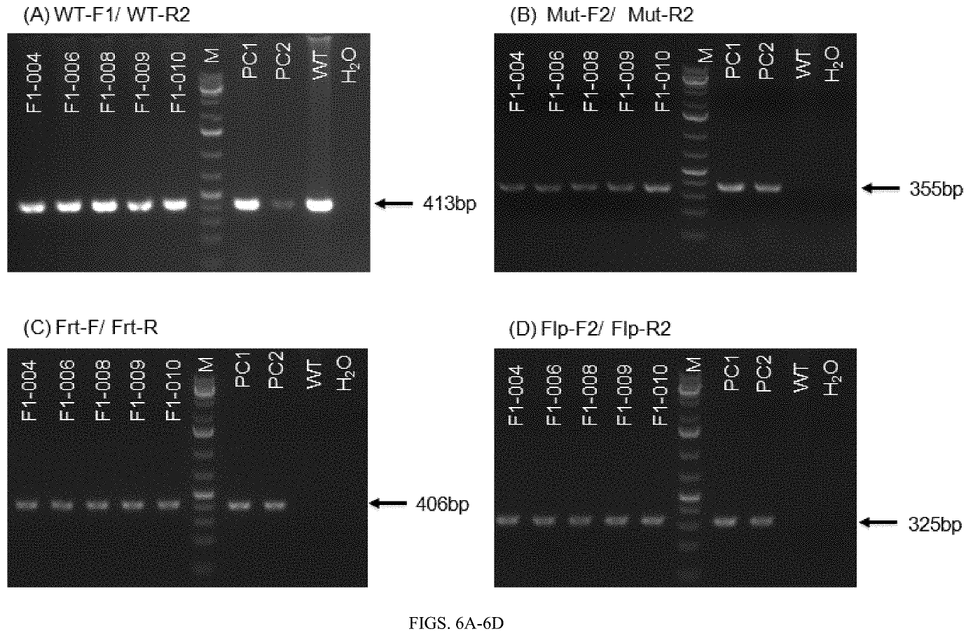

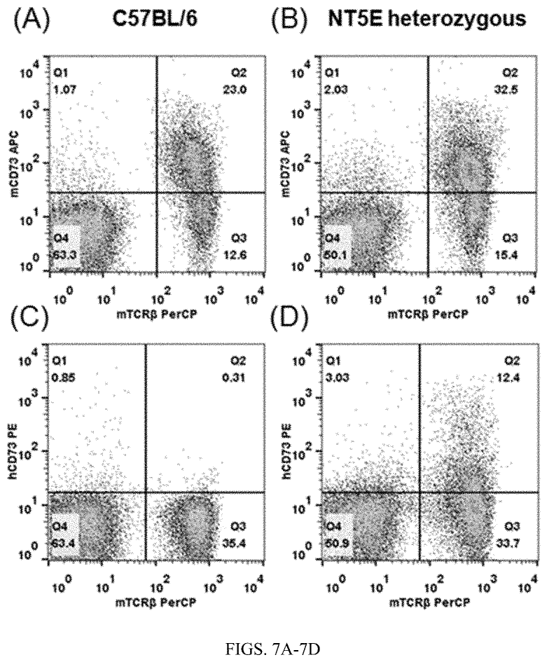

The positive clones that had been screened (black mice) were introduced into isolated blastocysts (white mice), and the obtained chimeric blastocysts were transferred to the culture medium for short-term culture and then transplanted to the fallopian tubes of the recipient mother (white mice) to produce the F0 chimeric mice (black and white). The F2 generation homozygous mice can be obtained by backcrossing the F0 generation chimeric mice with wild-type mice to obtain the F1 generation mice, and then mating the F1 generation heterozygous mice with each other. The positive mice were also mated with the Flp tool mice to remove the positive selectable marker gene (the schematic diagram of the process was shown in FIG. 5), and then the humanized NT5E homozygous mice expressing human CD73 protein can be obtained by mating with each other. The genotype of the progeny mice can be identified by PCR, and the results for the F1 generation mice (Neo-removed) are shown in FIGS. 6A-6D, wherein the four mice numbered F1-004, F1-006, F1-008, F1-009 were positive heterozygous mice.

The following primers were used in PCR:

| (SEQ ID NO: 21) | |
|---|---|
| WT-F1: | 5′- CTGCCCCTGCAGTTGTCACCG-3′, |
| (SEQ ID NO: 22) | |
| WT-R1: | 5′- AGCTCCCAGGCACTGGCTGCG-3′; |
| (SEQ ID NO: 23) | |
| Mut-F2: | 5′- TCCTGTTGGTGATGAAGTTGTGGGA-3′, |
| (SEQ ID NO: 24 | |
| Mut-R2: | 5′- AGCATAGGCCTGGACTACAGGAACC-3′; |
| (SEQ ID NO: 25) | |
| Frt-F: | 5′- CCTCAGACGAGTCGGATCTCCCTTT-3′, |
| (SEQ ID NO: 26) | |
| Frt-R: | 5′- CTGCGGGCCACTGTGGTAGTAGAG-3′; |
| (SEQ ID NO: 27) | |
| Flp-F: | 5′- GACAAGCGTTAGTAGGCACATATAC-3′, |
| (SEQ ID NO: 28) | |
| Flp-R: | 5′- GCTCCAATTTCCCACAACATTAGT-3′. |

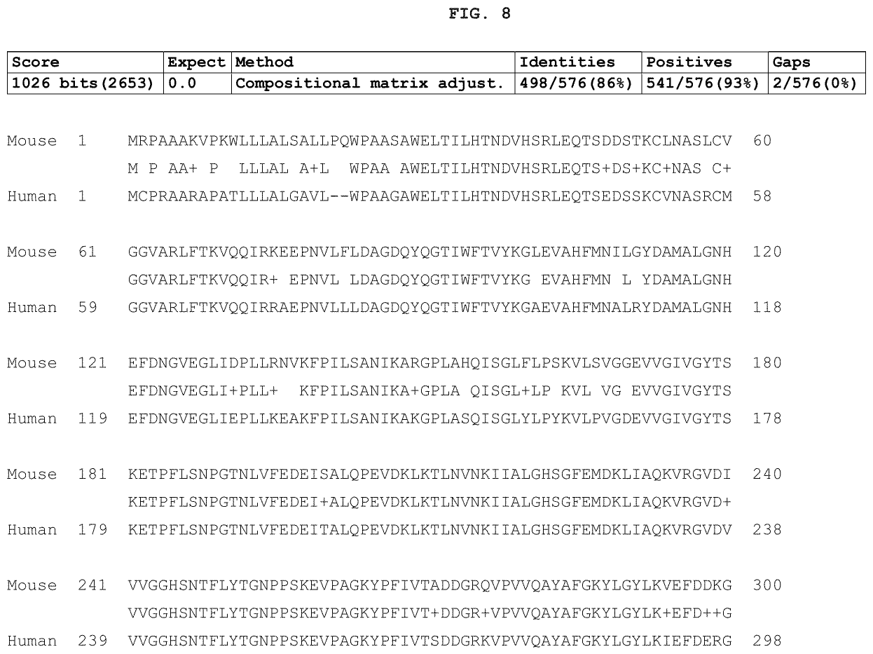

The expression of humanized CD73 protein in mice can be confirmed by routine detection methods. For example, wild-type C57BL/6 mice and NT5E gene humanized heterozygous mice were selected. CD73 protein expression were detected by staining the mouse spleen cells with (1) anti-mouse CD73 antibody (mCD73 APC) combined with murine T cell surface antibody PerCP/Cy55 Anti-mouse TCR Beta Chain Antibody (mTcRβ PerCP), or (2) anti-human CD73 antibody (hCD73 PE) combined with murine T cell surface antibody PerCP/Cy55 Anti-mouse TCR Beta Chain Antibody (mTcRβ PerCP). Flow cytometry analysis was performed. The results of the flow cytometry analysis (see FIGS. 7A-7D) showed that cells expressing mouse CD73 protein (FIG. 7B) and human CD73 protein were detected in the spleen of the humanized NT5E heterozygous mouse (FIG. 7D). In the spleen of the wild-type control mice, only the mouse CD73 protein was detected (FIG. 7A), and no cells expressing the human or humanized CD73 protein were detected (FIG. 7C).

### Example 2: Double-Gene Humanized or Multiple-Gene Humanized Mice

Mice with the humanized NT5E gene (e.g., animal model with human or chimeric NT5E prepared using the methods as described in the present disclosure) can also be used to prepare an animal model with double-humanized or multi-humanized genes. For example, the fertilized egg cells used in microinjection and embryo transfer can be selected from fertilized egg cells from other genetically modified mice. For example, PD-1 and NT5E double gene humanized mouse models can be obtained by gene editing of fertilized egg cells from PD-1 humanized mice using the methods described herein. In addition, the genetically engineered NT5E animal model homozygote or heterozygote can be mated with other genetically modified homozygous or heterozygous animal models (or through IVF). According to the Mendelian inheritance, there is a chance to obtain double-gene or multiple-gene modified heterozygous mice, and then the heterozygous animals can be further mated with each other to finally obtain the double-gene or multiple-gene modified homozygous mice.

For example, since the mouse CTLA4 gene and NT5E gene are located on chromosome 1 and chromosome 9 respectively, the double humanized CTLA4/NT5E mouse model can be obtained by mating the CTLA4 humanized mice with NT5E humanized mice. Double humanized CTLA4/NT5E gene mice can then be obtained by screening the progeny.

### Example 3: Pharmacological Validation of Humanized Animal Model

NT5E humanized mice can be used to evaluate the effects of modulators (e.g., agonists, antagonists, and inhibitors) targeting human CD73. For example, homozygous mice with humanized NT5E gene were subcutaneously injected with mouse colon cancer cell MC38, or MC38-hCD73 (MC38 cells expressing human CD73). When the tumor volume reached about 100 mm³, the mice were divided to a control group and a treatment group based on tumor size. The treatment group was treated with antibody MEDI9447 targeting human NT5E, and the control group was injected with an equal volume of hIgG (non-specific). The frequency of administration was twice a week for a total of 6 times. The euthanasia test was performed when the tumor volume of a single mouse reached 3000 mm³ after inoculation (Table 4).

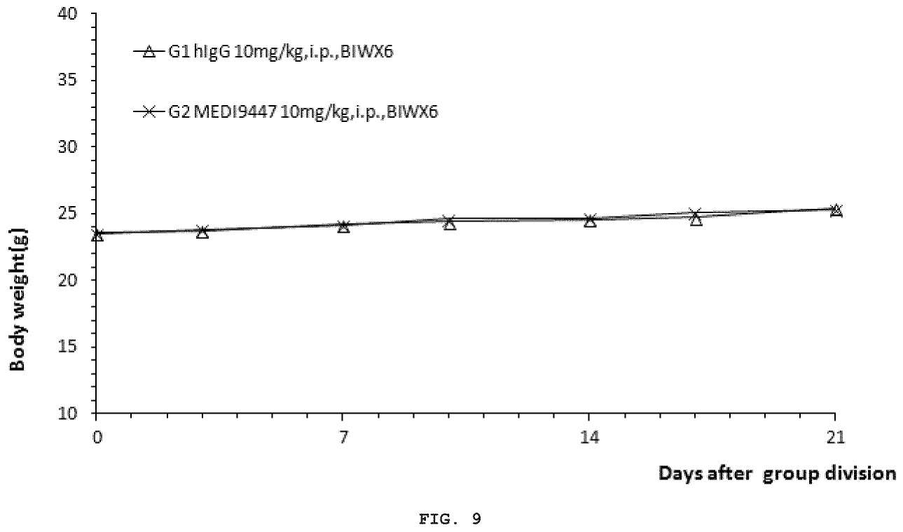

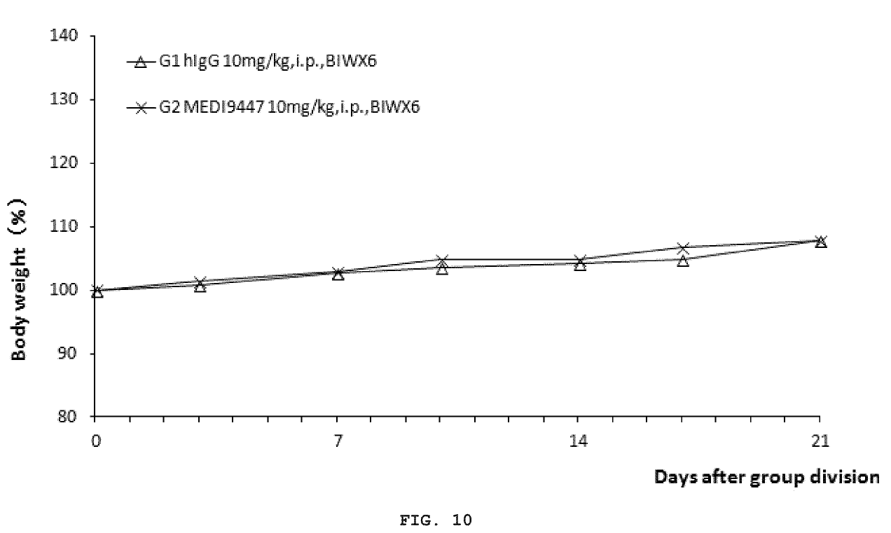

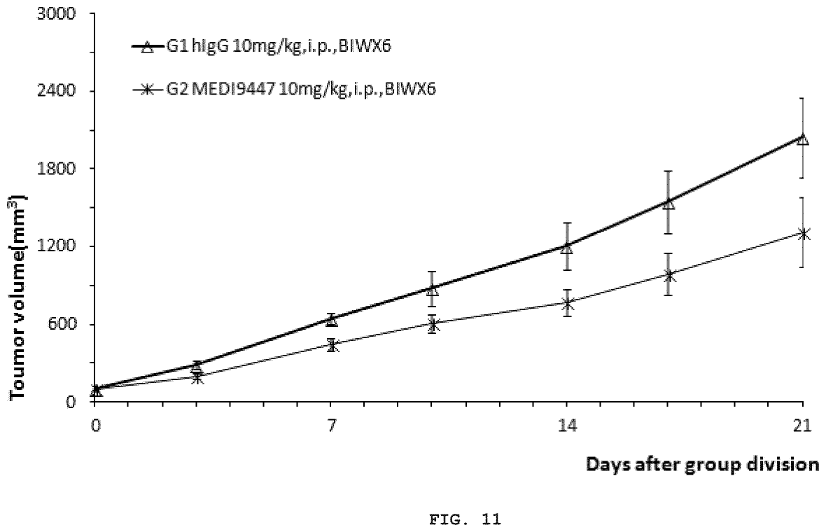

Tumor volume and the body weight were monitored to evaluate the in vivo toxicity and in vivo efficacy. During the experiment, the treatment groups and control groups were in good health. At the end point of the experiment, the body weight in all groups showed weight gain, and there was no significant difference between the treatment group and the control group (FIGS. 9-10) indicating that the animals tolerated MEDI9447 well. However, regarding tumor volumes (FIG. 11, Table 3), at the experimental endpoint, the average tumor volume of treatment groups was significantly smaller than the control group, indicating that the treatment with the human CD73 antibody MEDI9447 effectively inhibited the tumor growth in mouse. These results demonstrate that humanized CD73 animal models can be used to assess the efficacy of drugs targeting CD73 in vivo, as well as to assess the therapeutic efficacy of targeting CD73.

TABLE 3

| | | Tumor volume (mm3) | | | | | Non-existence | |
|---|---|---|---|---|---|---|---|---|
| | | Day 0 | Day 7 | Day 14 | Day 21 | Survival | of tumor | TGITV % |
| Control | G1 | 101 ± 3 | 643 ± 42 | 1202 ± 185 | 2046 ± 309 | 5/5 | 0/5 | N/A |
| Treatment | G2 | 100 ± 6 | 445 ± 51 | 767 ± 101 | 1311 ± 268 | 5/5 | 0/5 | 37.8 |

TABLE 4

| Group | Drug/compound | Dose/administration manner/frequency |
|---|---|---|
| G1 | hIgG | 10 mg/kg: intraperitoneal injection, |
| | | twice a week |
| G2 | MEDI9447 | 10 mg/kg: intraperitoneal injection, |
| | | twice a week |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A genetically-modified non-human animal, whose genome comprises at least one chromosome comprising a sequence encoding a human or chimeric CD73, wherein the sequence encoding the human or chimeric CD73 is operably linked to an endogenous promoter.

2. The animal of claim 1, wherein the human or chimeric CD73 comprises an amino acid sequence that is at least 70% identical to SEQ ID NO: 4 or SEQ ID NO: 31.

3. The animal of claim 1, wherein the sequence encoding a human or chimeric CD73 is operably linked to a Woodchuck Hepatitis Virus (WEEP) Posttranscriptional Regulatory Element or a polyA (polyadenylation) signal sequence.

4. (canceled)

5. The animal of claim 1, wherein the animal is a a rodent.

6. The animal of claim 1, wherein the animal is a mouse.

7. The animal of claim 1, wherein the animal does not express endogenous CD73 or expresses a decreased level of CD73 as compared to CD73 expression level in a wildtype animal.

8. The animal of claim 1, wherein the animal has one or more cells expressing human or chimeric CD73.

9. A genetically-modified, non-human animal, wherein the genome of the animal comprises an insertion of a sequence encoding a human CD73_or a chimeric CD73 at an endogenous CD73 gene locus.

10. The animal of claim 9, wherein the sequence encoding the human CD73 or the chimeric CD73 is operably linked to the 5′-UTR at the endogenous CD73 locus, and one or more cells of the animal express the human CD73 or the chimeric CD73.

11.-13. (canceled)

14. The animal of claim 9, wherein the animal is homozygous with respect to the insertion at the endogenous CD73 gene locus.

15.-18. (canceled)

19. A non-human animal comprising at least one cell comprising a nucleotide sequence encoding a human or chimeric CD73 polypeptide, wherein the human or chimeric CD73 polypeptide comprises at least 50 contiguous amino acid residues that are identical to the corresponding contiguous amino acid sequence of a human CD73, wherein the animal expresses the human or chimeric CD73 polypeptide.

20. The animal of claim 19, wherein the human or chimeric CD73 polypeptide comprises a sequence that is at least 80% identical to SEQ ID NO: 4 or SEQ ID NO: 31.

21. The animal of claim 19, wherein the nucleotide sequence is operably linked to the 5′-UTR at the endogenous CD73 locus immediately before the translation start codon.

22. The animal of claim 19, wherein the nucleotide sequence is integrated to an endogenous CD73 gene locus of the animal.

23.-24. (canceled)

25. The animal of claim 1, wherein the animal further comprises a sequence encoding an additional human or chimeric protein.

26. The animal of claim 25, wherein the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD3, CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α_(SIRPα) or TNF Receptor Superfamily Member 4 (OX40).

27.-28. (canceled)

29. A method of determining effectiveness of an anti-CD73 antibody for the treatment of cancer, comprising:
administering the anti-CD73 antibody to the animal of claim 1, wherein the animal has a tumor; and
determining the inhibitory effects of the anti-CD73 antibody to the tumor.

30. (canceled)

31. The method of claim 29, wherein the tumor comprises one or more cancer cells that are injected into the animal.

32. The method of claim 29, wherein determining the inhibitory effects of the anti-CD73 antibody to the tumor involves measuring the tumor volume in the animal.

33. The method of claim 29, wherein the animal has a solid tumor, glioma, head and neck cancer, melanoma, thyroid cancer, breast cancer, pancreatic cancer, colon cancer, bladder cancer, ovarian cancer, prostate cancer, or leukemia.

34.-41. (canceled)
